(19) United States
(12) Patent Application Publication
(10) Pub. No.: US 2010/0093722 A1
(43) Pub. Date: Apr. 15, 2010

(54) HETEROARYL AND PHENYL SUBSTITUTED THIENO[2,3-d]PYRIMIDINES AND THEIR USE AS ADENOSINE A2a RECEPTOR ANTAGONISTS

(76) Inventors: J. Kent BARBAY, Flourtown, PA (US); Devraj Chakravarty, Hillsborough, NJ (US); Kristi Leonard, Flourtown, PA (US); Brian Christopher Shook, Gilbertsville, PA (US); Aihua Wang, Jamison, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/499,342

(22) Filed: Jul. 8, 2009

Related U.S. Application Data
(60) Provisional application No. 61/104,786, filed on Oct. 13, 2008.

(51) Int. Cl. A61K 31/519; C07D 495/04; A61K 31/5377; A61P 25/00

(52) U.S. Cl. 514/234.2; 544/278; 544/117; 514/260.1; 514/255.05

(57) ABSTRACT

This invention relates to a novel thieno[2,3-d]pyrimidine, Z, and its therapeutic and prophylactic uses, wherein R¹ and R² are defined in the specification. Disorders treated and/or prevented include Parkinson's Disease.

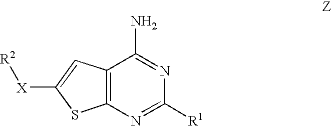

### CROSS-REFERENCE TO RELATED APPLICATIONS

The present application claims the benefits of the filing of U.S. Provisional Application No. 61/104,786 filed Oct. 13, 2008. The complete disclosures of the aforementioned related patent applications are hereby incorporated herein by reference for all purposes.

### FIELD OF THE INVENTION

This invention relates to a novel arylindenopyrimidine and its therapeutic and prophylactic uses. Disorders treated and/or prevented include neurodegenerative and movement disorders ameliorated by antagonizing Adenosine A2a receptors.

### BACKGROUND OF THE INVENTION

Adenosine A2a Receptors Adenosine is a purine nucleotide produced by all metabolically active cells within the body. Adenosine exerts its effects via four subtypes of cell surface receptors (A1, A2a, A2b and A3), which belong to the G protein coupled receptor superfamily (Stiles, G. L. Journal of Biological Chemistry, 1992, 267, 6451). A1 and A3 couple to inhibitory G protein, while A2a and A2b couple to stimulatory G protein. A2a receptors are mainly found in the brain, both in neurons and glial cells (highest level in the striatum and nucleus accumbens, moderate to high level in olfactory tubercle, hypothalamus, and hippocampus etc. regions) (Rosin, D. L.; Robeva, A.; Woodard, R. L.; Guyenet, P. G.; Linden, J. Journal of Comparative Neurology, 1998, 401, 163).

In peripheral tissues, A2a receptors are found in platelets, neutrophils, vascular smooth muscle and endothelium (Gessi, S.; Varani, K.; Merighi, S.; Ongini, E.; Bores, P. A. British Journal of Pharmacology, 2000, 129, 2). The striatum is the main brain region for the regulation of motor activity, particularly through its innervation from dopaminergic neurons originating in the substantial nigra. The striatum is the major target of the dopaminergic neuron degeneration in patients with Parkinson's Disease (PD). Within the striatum, A2a receptors are co-localized with dopamine D2 receptors, suggesting an important site for the integration of adenosine and dopamine signaling in the brain (Fink, J. S.; Weaver, D. Ri; Rivkees, S. A.; Peterfreund, R. A.; Pollack, A. E.; Adler, E. M.; Reppert, S. M. Brain Research Molecular Brain Research, 1992, 14, 186).

Neurochemical studies have shown that activation of A2a receptors reduces the binding affinity of D2 agonist to their receptors. This D2R and A2aR receptor-receptor interaction has been demonstrated instriatal membrane preparations of rats (Ferre, S.; con Euler, G.; Johansson, B.; Fredholm, B. B.; Fuxe, K. Proceedings of the National Academy of Sciences I of the United States of America, 1991, 88, 7238) as well as in fibroblast cell lines after transfected with A2aR and D2R cDNAs (Salim, H.; Ferre, S.; Dalal, A.; Peterfreund, R. A.; Fuxe, K.; Vincent, J. D.; Lledo, P. M. Journal of Neurochemistry, 2000, 74, 432). In vivo, pharmacological blockade of A2a receptors using A2a antagonist leads to beneficial effects in dopaminergic neurotoxin MPTP (1-methyl-4-pheny-1,2,3,6-tetrahydropyridine)-induced PC) in various species, including mice, rats, and monkeys (Ikeda, K.; Kurokawa, M.; Aoyana, S.; Kuwana, Y. Journal of Neurochemistry, 2002, 80, 262).

Furthermore, A2a knockout mice with genetic blockade of A2a function have been found to be less sensitive to motor impairment and neurochemical changes when they were exposed to neurotoxin MPTP (Chen, J. F.; Xu, K.; I Petzer, J. P.; Steal, R.; Xu, Y. H.; Beilstein, M.; Sonsalla, P. K.; Castagnoli, K.; Castagnoli, N., Jr.; Schwarsschild, M. A. Journal of Neuroscience, 2001, 121, RC143).

In humans, the adenosine receptor antagonist theophylline has been found to produce beneficial effects in Parkinson's disease patients (Mally, J.; Stone, T. W. Journal of the Neurological Sciences, 1995, 132, 129). Consistently, recent epidemiological study has shown that high caffeine consumption makes people less likely to develop PD (Ascherio, A.; Zhang, S. M.; Hernan, M. A.; Kawachi, I.; Colditz, G. A.; Speizer, F. E.; Willett, W. C. Annals of Neurology, 2001, 50, 56). In summary, adenosine A2a receptor blockers may provide a new class of antiparkinsonian agents (Impagnatiello, F.; Bastia, E.; Ongini, E.; Monopoli, A. Emerging Therapeutic Targets, 2000, 4, 635).

Antagonists of the A_{2A} receptor are potentially useful therapies for the treatment of addiction. Major drugs of abuse (opiates, cocaine, ethanol, and the like) either directly or indirectly modulate dopamine signaling in neurons particularly those found in the nucleus accumbens, which contain high levels of A_{2A} adenosine receptors. Dependence has been shown to be augmented by the adenosine signaling pathway, and it has been shown that administration of an A_{2A} receptor antagonist reduces the craving for addictive substances (“The Critical Role of Adenosine A_{2A} Receptors and Gi βγ Subunits in Alcoholism and Addiction: From Cell Biology to Behavior”, by Ivan Diamond and Lina Yao, (The Cell Biology of Addiction, 2006, pp 291-316) and “Adaptations in Adenosine Signaling in Drug Dependence: Therapeutic Implications”, by Stephen P. Hack and Macdonald J. Christie, Critical Review in Neurobiology, Vol. 15, 235-274 (2003)). See also Alcoholism: Clinical and Experimental Research (2007), 31(8), 1302-1307.

An A_{2A} receptor antagonist could be used to treat attention deficit hyperactivity disorder (ADHD) since caffeine (a non selective adenosine antagonist) can be useful for treating ADHD, and there are many interactions between dopamine and adenosine neurons. Clinical Genetics (2000), 58(1), 31-40 and references therein.

Antagonists of the A_{2A} receptor are potentially useful therapies for the treatment of depression. A_{2A} antagonists are known to induce activity in various models of depression including the forced swim and tail suspension tests. The positive response is mediated by dopaminergic transmission and is caused by a prolongation of escape-directed behavior rather than by a motor stimulant effect. Neurology (2003), 61(suppl 6) S82-S87.

Antagonists of the A_{2A} receptor are potentially useful therapies for the treatment of anxiety. A_{2A} antagonist have been shown to prevent emotional/anxious responses in vivo. Neurobiology of Disease (2007), 28(2) 197-205.

### SUMMARY OF THE INVENTION

The present invention includes compounds of Formula Z

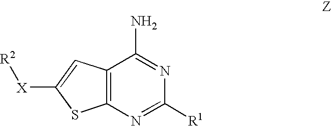

wherein:

X is selected from the group consisting of:

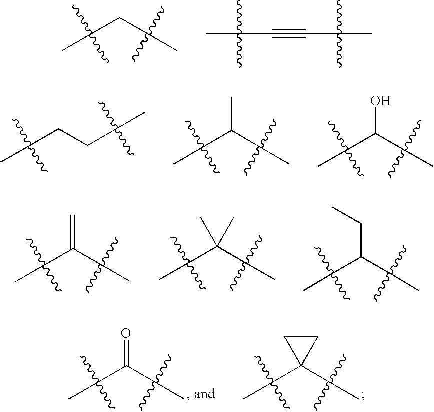

R¹ is phenyl wherein said phenyl is optionally substituted with up to three substituents independently selected from the group consisting of F, Cl, Br, and OCH₃, or a single substituent selected from the group consisting of: OH, OCH₂CF₃, OC₍₁₋₄₎alkyl, C₍₁₋₄₎alkyl, CHF₂, OCF₃, CF₃, and CN;

R² is heteroaryl wherein said heteroaryl is optionally substituted with Cl, F, Br, OC₍₁₋₄₎alkyl, OCF₃, OH, C₍₁₋₄₎alkyl, CHF₂, CF₃, OCH₂CF₃, or a ring selected from the group consisting of:

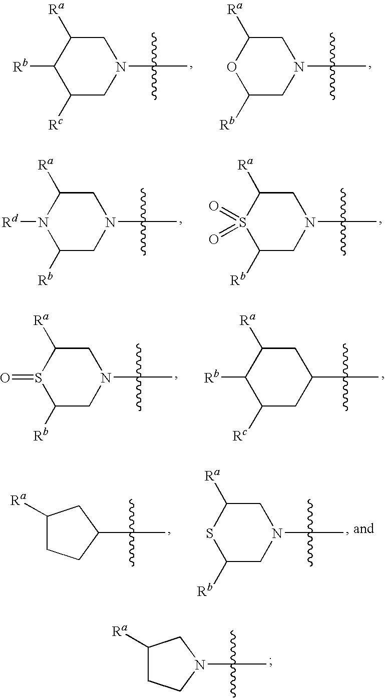

wherein R^{a}, R^{b}, and R^{c} are independently H or C₍₁₋₄₎alkyl;
R^{d} is H, —C₍₁₋₄₎alkyl, —CH₂CH₂OCH₂OCH₃, —CH₂CO₂H, —C(O)C₍₁₋₄₎alkyl, or —CH₂C(O)C₍₁₋₄₎alkyl;

and solvates, hydrates, tautomers and pharmaceutically acceptable salts thereof.

### DETAILED DESCRIPTION OF THE INVENTION

The present invention includes compounds of Formula Z

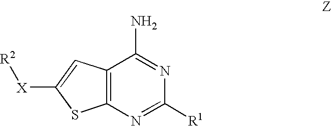

wherein:

X is selected from the group consisting of:

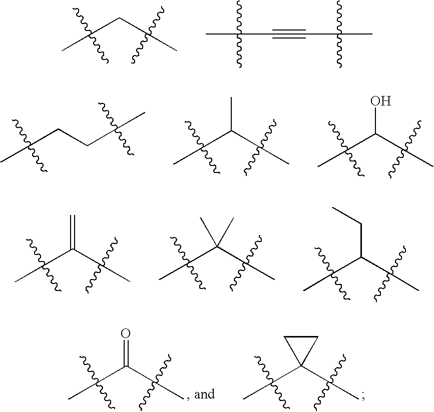

R¹ is phenyl wherein said phenyl is optionally substituted with up to three substituents independently selected from the group consisting of F, Cl, Br, and OCH₃, or a single substituent selected from the group consisting of: OH, OCH₂CF₃, OC₍₁₋₄₎alkyl, C₍₁₋₄₎alkyl, CHF₂, OCF₃, CF₃, and CN;

R² is heteroaryl wherein said heteroaryl is optionally substituted with Cl, F, Br, OC₍₁₋₄₎alkyl, OCF₃, OH, C₍₁₋₄₎alkyl, CHF₂, CF₃, OCH₂CF₃, or a ring selected from the group consisting of:

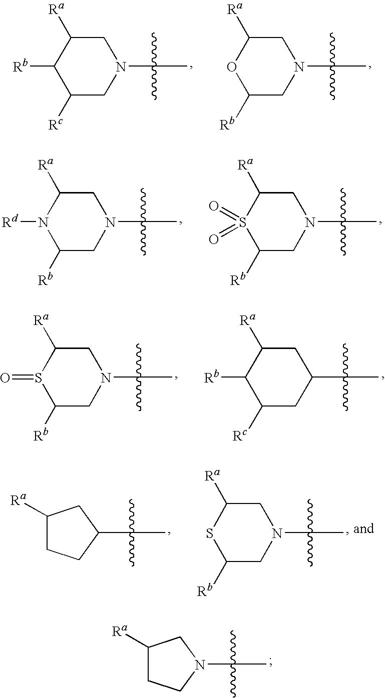

wherein R^{a}, R^{b}, and R^{c} are independently H or C₍₁₋₄₎alkyl;
R^{d} is H, —C₍₁₋₄₎alkyl, —CH₂CH₂OCH₂CH₂OCH₃, —CH₂CO₂H, —C(O)C₍₁₋₄₎alkyl, or —CH₂C(O)C₍₁₋₄₎alkyl;

and solvates, hydrates, tautomers and pharmaceutically acceptable salts thereof.

In another embodiment of the invention:

X is selected from the group consisting of:

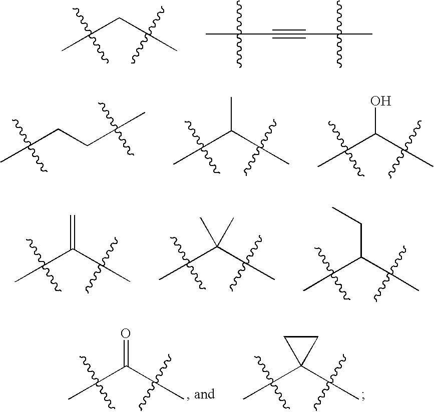

R¹ is phenyl, optionally substituted with CN, CF₃, OC₍₁₋₄₎alkyl, OCF₃, C₍₁₋₄₎alkyl, OCH₂CF₃, or up to 3 halogens, selected from the group consisting of Cl, and F;

R² is selected from the group consisting of furyl, imidazolyl, pyrrolyl, oxazolyl, isoxazolyl, pyridinyl, pyrimidinyl, and pyridazinyl, wherein said pyridinyl, pyrimidinyl, and pyridazinyl are optionally substituted with Cl, F, Br, OC₍₁₋₄₎alkyl, OCF₃, piperidinyl, 6-methylpiperidinyl, 3-methylpyrrolidinyl, pyrrolidinyl, morpholinyl, or OCH₂CF₃:

and solvates, hydrates, tautomers and pharmaceutically acceptable salts thereof.

In another embodiment of the invention:

X is selected from the group consisting of:

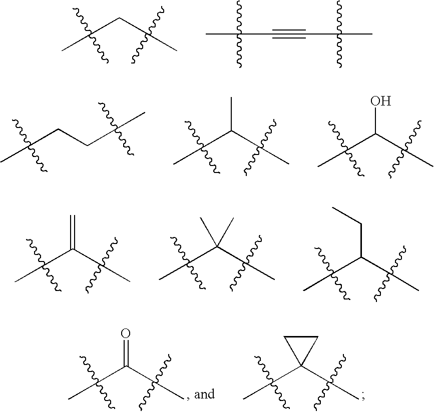

R¹ is phenyl, optionally substituted with CN, CF₃, OC₍₁₋₄₎alkyl, OCF₃, C₍₁₋₄₎alkyl, or up to 3 halogens, selected from the group consisting of Cl, and F;

R² is selected from the group consisting of pyridinyl, pyrimidinyl, and pyridazinyl, wherein said pyridinyl, pyrimidinyl, and pyridazinyl are optionally substituted with Cl, F, Br, OC₍₁₋₄₎alkyl, piperidinyl, pyrrolidinyl, morpholinyl, or OCH₂CF₃:

and solvates, hydrates, tautomers and pharmaceutically acceptable salts thereof.

In another embodiment of the invention:

X is selected from the group consisting of:

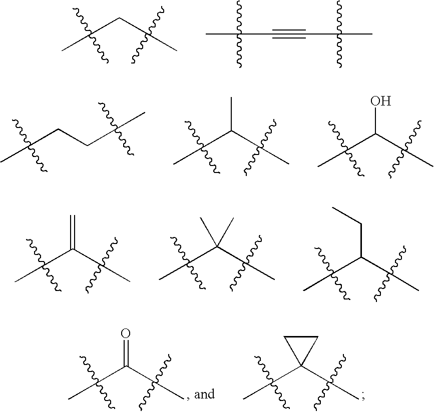

R¹ is phenyl, optionally substituted with CN, CF₃, or up to 3 halogens, selected from the group consisting of Cl, and F;

R² is selected from the group consisting of pyridinyl, and pyridazinyl, wherein said pyridinyl is optionally substituted with Cl, F, Br, OC₍₁₋₄₎alkyl, piperidinyl, pyrrolidinyl, morpholinyl, or OCH₂CF₃:

and solvates, hydrates, tautomers and pharmaceutically acceptable salts thereof.

In another embodiment of the invention:

X is selected from the group consisting of:

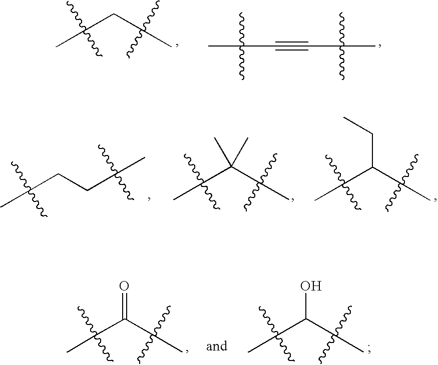

R¹ is phenyl, optionally substituted with CN, or F;

R² is selected from the group consisting of:

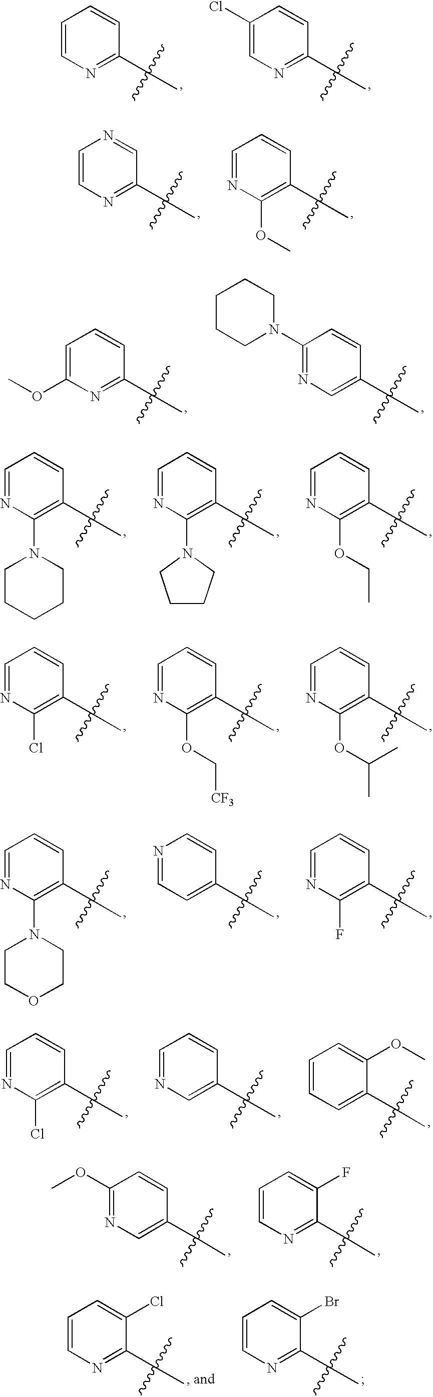

and solvates, hydrates, tautomers and pharmaceutically acceptable salts thereof.

Another embodiment of the invention comprises a compound selected from the group consisting of:

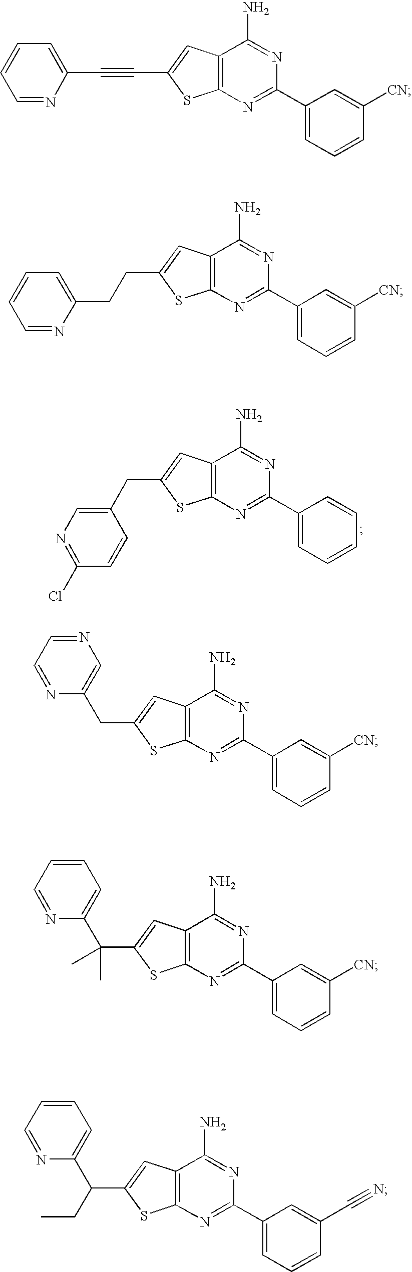

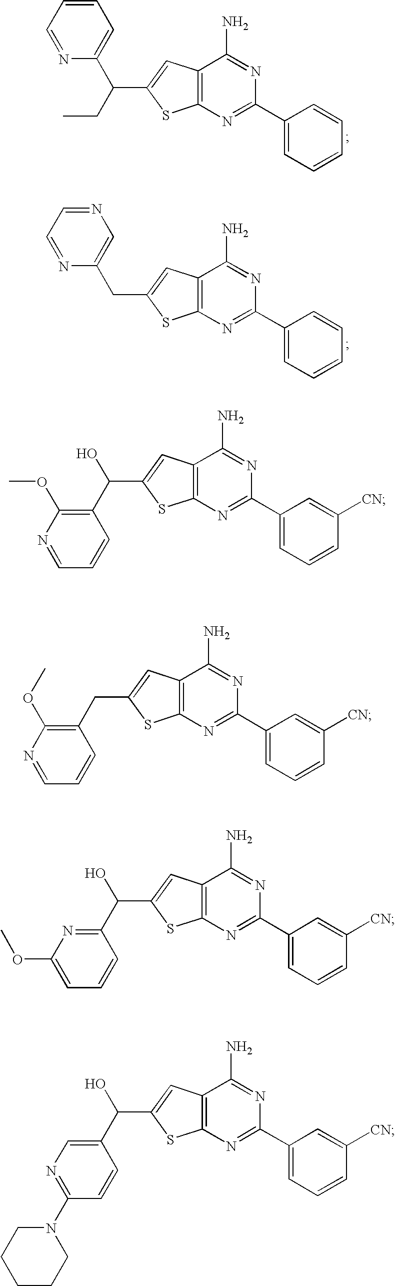

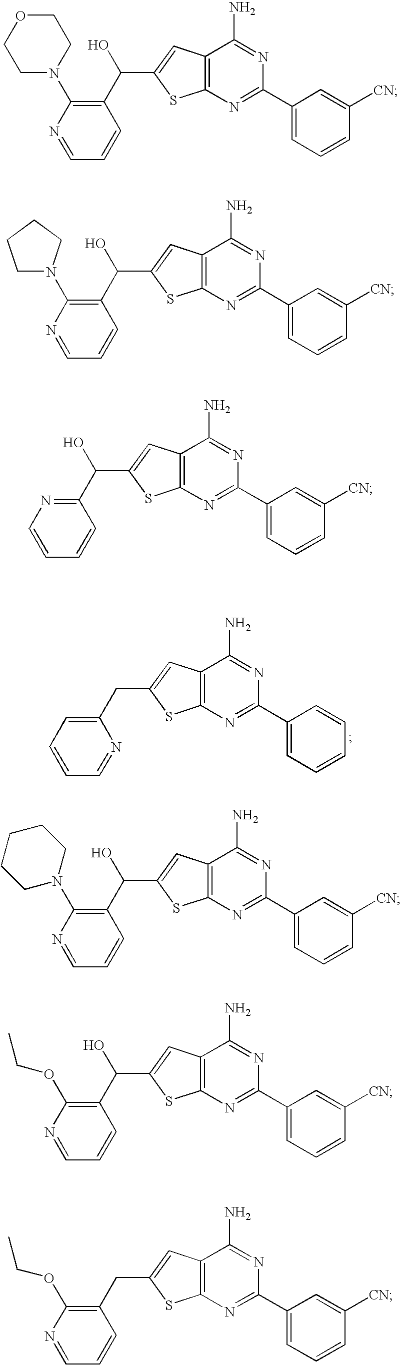

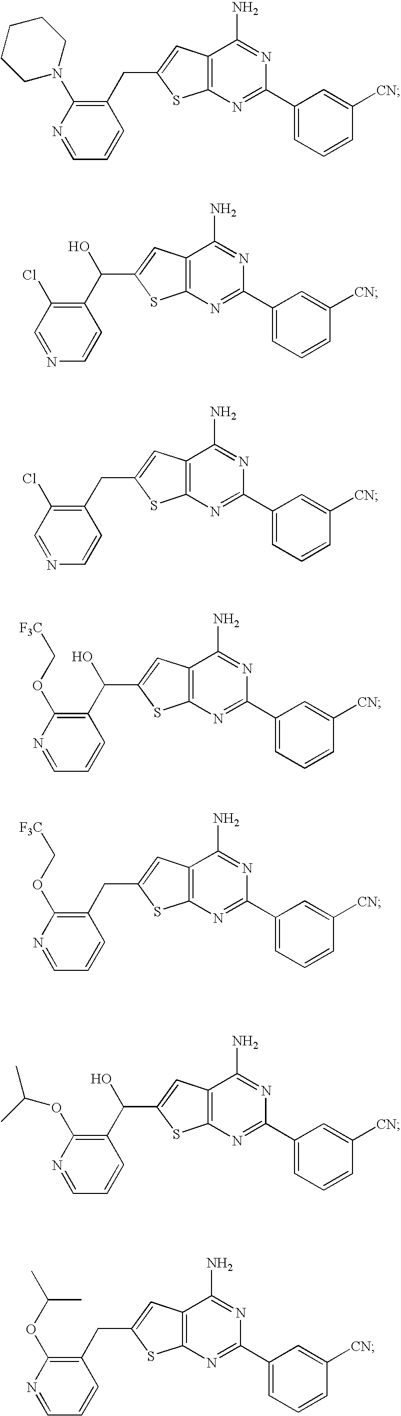

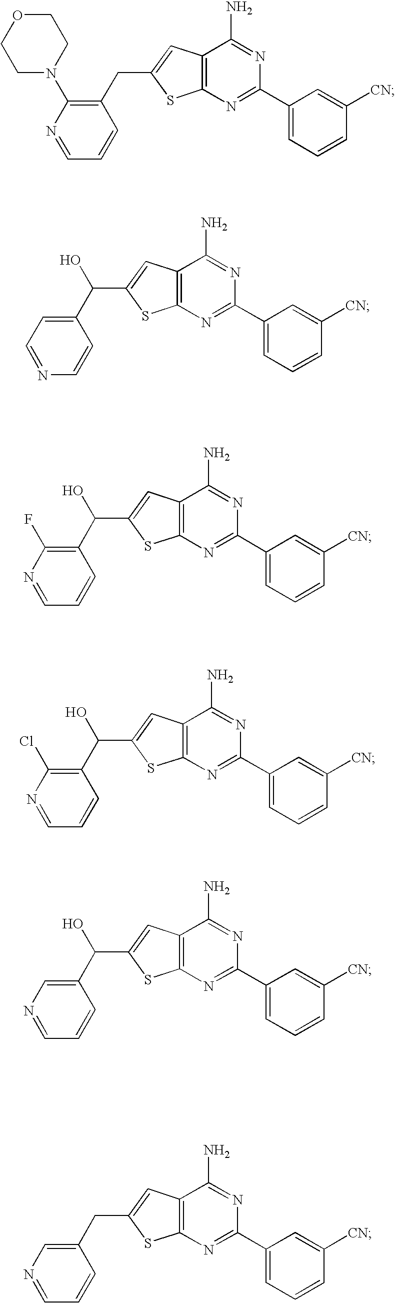

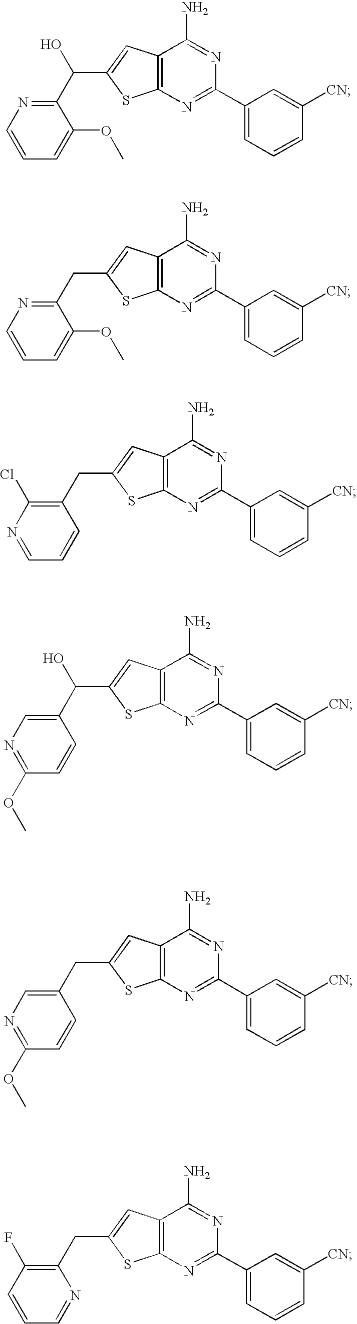

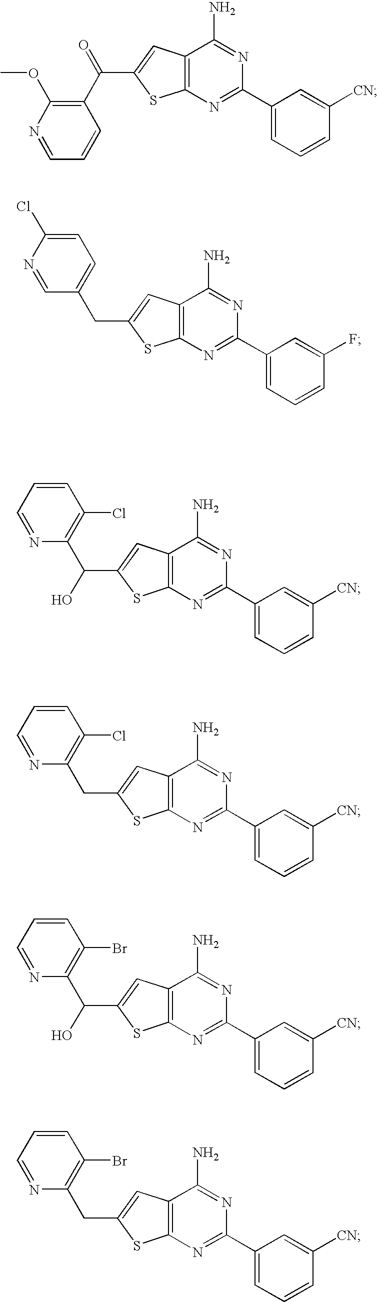

and solvates, hydrates, tautomers and pharmaceutically acceptable salts thereof.

This invention further provides a method of treating a subject having a condition ameliorated by antagonizing Adenosine A2a receptors, which comprises administering to the subject a therapeutically effective dose of a compound of Formula Z.

This invention further provides a method of preventing a disorder ameliorated by antagonizing Adenosine A2a receptors in a subject, comprising of administering to the subject a prophylactically effective dose of the compound of claim 1 either preceding or subsequent to an event anticipated to cause a disorder ameliorated by antagonizing Adenosine A2a receptors in the subject.

Compounds of Formula Z can be isolated and used as free bases. They can also be isolated and used as pharmaceutically acceptable salts.

Examples of such salts include hydrobromic, hydroiodic, hydrochloric, perchloric, sulfuric, maleic, fumaric, malic, tartaric, citric, adipic, benzoic, mandelic, methanesulfonic, hydroethanesulfonic, benzenesulfonic, oxalic, palmoic, 2 naphthalenesulfonic, p-toluenesulfonic, cyclohexanesulfamic and saccharinc

This invention also provides a pharmaceutical composition comprising a compound of Formula Z and a pharmaceutically acceptable carrier.

Pharmaceutically acceptable carriers are well known to those skilled in the art and include, but are not limited to, from about 0.01 to about 0.1 M and preferably 0.05 M phosphate buyer or 0.8% saline. Such pharmaceutically acceptable carriers can be aqueous or non-aqueous solutions, suspensions and emulsions. Examples of non-aqueous solvents are propylene glycol, polyethylene glycol, vegetable oils such as olive oil, and injectable organic esters such as ethyl oleate. Aqueous carriers include water, ethanol, alcoholic/aqueous solutions, glycerol, emulsions or suspensions, including saline and buffered media. Oral carriers can be elixirs, syrups, capsules, tablets and the like. The typical solid carrier is an inert substance such as lactose, starch, glucose, methyl-cellulose, magnesium stearate, dicalcium phosphate, mannitol and the like. Parenteral carriers include sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like.

Preservatives and other additives can also be present, such as, for example, antimicrobials, antioxidants, chelating agents, inert gases and the like. All carriers can be mixed as needed with disintegrants, diluents, granulating agents, lubricants, binders and the like using conventional techniques known in the art.

This invention further provides a method of treating a subject having a condition ameliorated by antagonizing Adenosine A2a receptors, which comprises administering to the subject a therapeutically effective dose of a compound of Formula Z.

In one embodiment, the disorder is a neurodegenerative or movement disorder. Examples of disorders treatable by the instant pharmaceutical composition include, without limitation, Parkinson's Disease, Huntington's Disease, Multiple System Atrophy, Corticobasal Degeneration, Alzheimer's Disease, and Senile Dementia.

In one preferred embodiment, the disorder is Parkinson's disease.

As used herein, the term “subject” includes, without limitation, any animal or artificially modified animal having a disorder ameliorated by antagonizing adenosine A2a receptors. In a preferred embodiment, the subject is a human.

Administering the instant pharmaceutical composition can be effected or performed using any of the various methods known to those skilled in the art. Compounds of Formula Z can be administered, for example, intravenously, intramuscularly, orally and subcutaneously. In the preferred embodiment, the instant pharmaceutical composition is administered orally. Additionally, administration can comprise giving the subject a plurality of dosages over a suitable period of time. Such administration regimens can be determined according to routine methods.

As used herein, a “therapeutically effective dose” of a pharmaceutical composition is an amount sufficient to stop, reverse or reduce the progression of a disorder. A “prophylactically effective dose” of a pharmaceutical composition is an amount sufficient to prevent a disorder, i.e., eliminate, ameliorate and/or delay the disorder's onset. Methods are known in the art for determining therapeutically and prophylactically effective doses for the instant pharmaceutical composition. The effective dose for administering the pharmaceutical composition to a human, for example, can be determined mathematically from the results of animal studies.

In one embodiment, the therapeutically and/or prophylactically effective dose is a dose sufficient to deliver from about 0.001 mg/kg of body weight to about 200 mg/kg of body weight of a compound of Formula Z. In another embodiment, the therapeutically and/or prophylactically effective dose is a dose sufficient to deliver from about 0.05 mg/kg of body weight to about 50 mg/kg of body weight. More specifically, in one embodiment, oral doses range from about 0.05 mg/kg to about 100 mg/kg daily. In another embodiment, oral doses range from about 0.05 mg/kg to about 50 mg/kg daily, and in a further embodiment, from about 0.05 mg/kg to about 20 mg/kg daily. In yet another embodiment, infusion doses range from about 1.0 ,ug/kg/min to about 10 mg/kg/min of inhibitor, admixed with a pharmaceutical carrier over a period ranging from about several minutes to about several days. In a further embodiment, for topical administration, the instant compound can be combined with a pharmaceutical carrier at a drug/carrier ratio of from about 0.001 to about 0.1.

The invention also provides a method of treating addiction in a mammal, comprising administering a therapeutically effective dose of a compound of Formula Z.

The invention also provides a method of treating ADHD in a mammal, comprising administering a therapeutically effective dose of a compound of Formula Z.

The invention also provides a method of treating depression in a mammal, comprising administering a therapeutically effective dose of a compound of Formula Z.

The invention also provides a method of treating anxiety in a mammal, comprising administering a therapeutically effective dose of a compound of Formula Z.

Definitions:

The term “C_{a-b}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₁₋₄ denotes a radical containing 1, 2, 3 or 4 carbon atoms.

The term “alkyl,” whether used alone or as part of a substituent group, refers to a saturated branched or straight chain monovalent hydrocarbon radical, wherein the radical is derived by the removal of one hydrogen atom from a single carbon atom. Unless specifically indicated (e.g. by the use of a limiting term such as “terminal carbon atom”), substituent variables may be placed on any carbon chain atom. Typical alkyl radicals include, but are not limited to, methyl, ethyl, propyl, isopropyl and the like. Examples include C₁₋₈alkyl, C₁₋₆alkyl and C₁₋₄alkyl groups.

The term “heteroaryl” refers to a radical derived by the removal of one hydrogen atom from a ring carbon atom of a heteroaromatic ring system. Typical heteroaryl radicals include furyl, pyrrolyl, oxazolyl, thiophenyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolizinyl, indolyl, isoindolyl, indazolyl, benzimidazolyl, benzothiazolyl, purinyl, 4H-quinolizinyl, quinolinyl, isoquinolinyl, cinnolinyl, phthalzinyl, quinazolinyl, quinoxalinyl, 1,8-naphthyridinyl, and pteridinyl.

The term “heterocyclyl” refers to a radical derived by the removal of one hydrogen atom from a ring carbon or ring nitrogen atom of a saturated or partially saturated heteroaromatic ring system. Typical heterocyclyl radicals include morpholinyl, piperidinyl, piperazinyl, pyrrolidinyl, and tetrahydrofuranyl.

Abbreviations:

Herein and throughout this application, the following abbreviations may be used.

BOC butyloxycarbonyl

n-BuLi n-butyllithium

t-BuOK potassium tert-butoxide

DMF dimethylformamide

DMAP dimethylaminopyridine

DMSO dimethylsulfoxide

Et ethyl

LDA lithium diisopropylamine

Me methyl

NBS N-bromo succinimide

NMO N-methylmorpholine-N-oxide

OAc acetate

Pd(dppf)Cl₂ [1,1′-Bis(diphenylphosphino)ferrocene]dichloropalladium (II)

Ph phenyl

TFA trifluoroacetic acid

THF tetrahydrofuran

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, Ed. H. Bundgaard, Elsevier, 1985.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

General Schemes:

Compounds of Formula Z can be prepared by methods known to those who are skilled in the art. The following reaction schemes are only meant to represent examples of the invention and are in no way meant to be a limit of the invention.

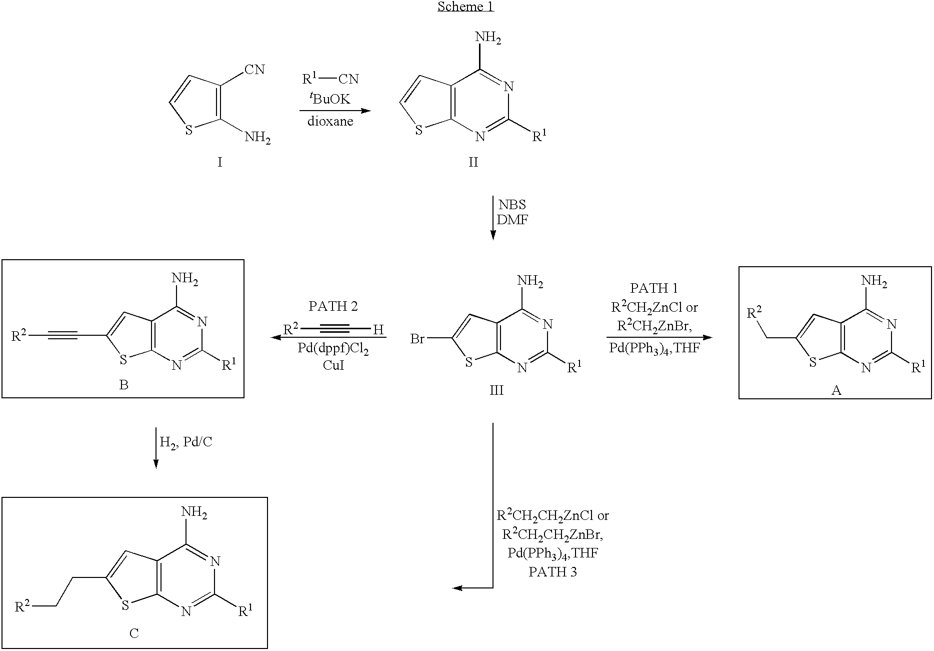

Scheme 1 illustrates the synthetic routes (Paths 1 and 2) leading to compounds of Formula Z (A, B, C). Starting with 2-amino-3-cyanothiophene I and following the path indicated by the arrows, condensation under basic conditions with R¹—CN, where R¹ is as defined in Formula Z, affords the aminopyrimidine II. The aminopyrimidine II is reacted with N-bromosuccinimide (NBS), to give the bromothiophene III. Following path 1 bromothiophene III is reacted with R²CH₂ZnCl or R²CH₂ZnBr, where R² is as defined in Formula Z, in the presence of a palladium catalyst to afford compounds of Formula Z, where X is CH₂ (A). Following path 2, bromothiophene III is reacted with R²CCH, where R² is as defined in Formula Z, in the presence of a palladium catalyst to give compounds of Formula Z, where X is

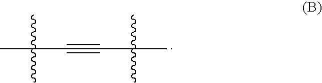

Compounds of Formula B can be reduced by hydrogenation to give compounds of Formula Z, where X is

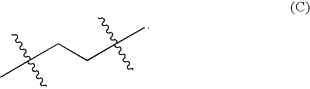

Alternatively, compounds of Formula C may be obtained using the procedure outlined in path 3. An bromothiophene III is reacted with R²CH₂CH₂ZnCl or R²CH₂CH₂ZnBr in the presence of a palladium catalyst to give compounds of Formula Z, where X is

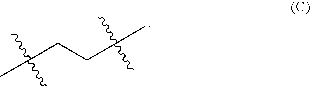

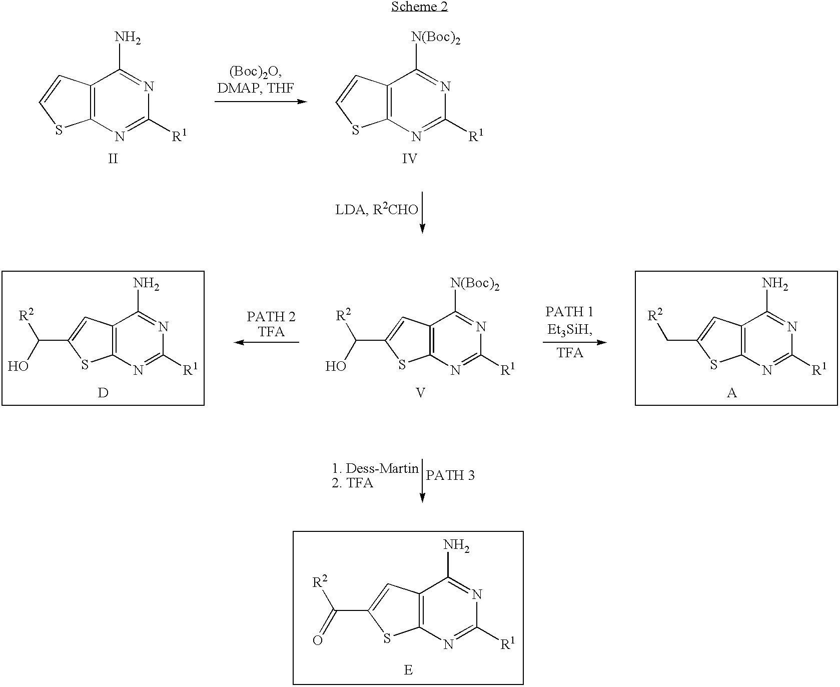

Scheme 2 illustrates the synthetic routes (Paths 1, 2 and 3) leading to compounds of Formula Z (A, D, E). Starting with aminopyrimidine II, prepared as described in Scheme 1, and following the path indicated by the arrows, reaction with di-tert-butyldicarbonate [(Boc)₂O] in the presence of 4-dimethylamino pyridine (DMAP) gives the corresponding protected amine IV. The thiophene IV is deprotonated with lithium diisopropylamide (LDA) and reacted with R²CHO, where R² is as defined in Formula Z, to give an intermediate alcohol V. Following path 1 the alcohol in V is reduced to the corresponding methylene using triethylsilane in trifluoroacetic acid (TFA) to give compounds of the Formula A. Following path 2, compound V is deprotected with TFA to give compounds of Formula D. Following path 3, V is oxidized using Dess-Martin reagent followed by deprotection with TFA to give compound of the Formula E.

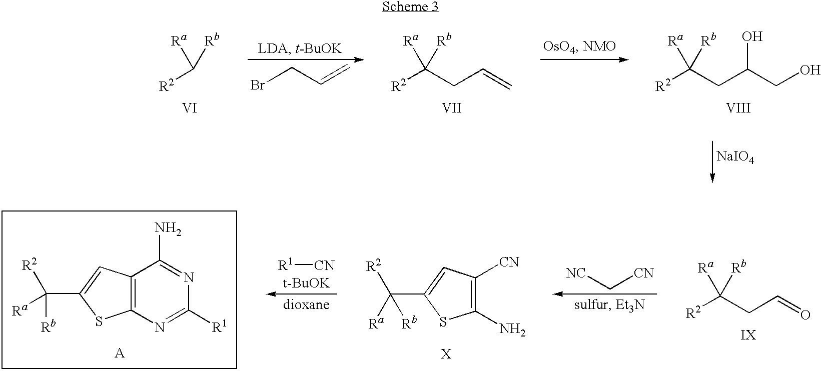

wherein R^{a}, R^{b} are independently selected from H, and CH₃, or R^{a} is H, and R^{b} is CH₂CH₃;

Scheme 3 illustrates the synthetic route leading to compounds of Formula A and alkyl substituted compounds of Formula A. Compound VI, where R² is as defined as in Formula Z, is deprotonated with LDA and potassium tert-butoxide (t-BuOK) and reacted with allyl bromide to give compound VII. Alkene VII is dihydroxylated with osmium tetroxide in the presence of N-methylmorpholine-N-oxide (NMO) to give the diol VIII. Diol VIII is reacted with sodium periodate to give the aldehyde IX. Aldehyde IX is reacted with malononitrile and elemental sulfur under basic conditions to give the thiophene X. The thiophene X is condensed under basic conditions with R¹—CN, where R¹ is as defined as in Formula Z, to afford compounds of Formula Z where X is CH₂ wherein said CH₂ is optionally substituted with C₍₁₋₂₎alkyl (A).

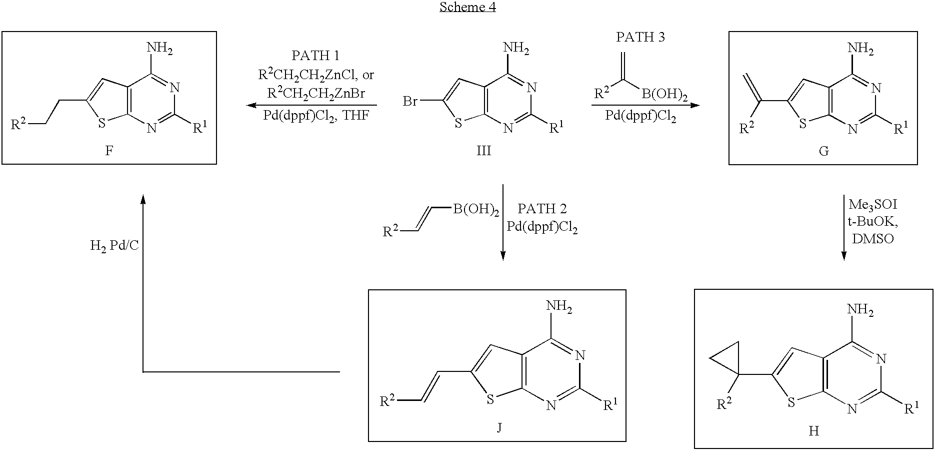

Scheme 4 illustrates the synthetic routes (Paths 1, 2 and 3) leading to compounds of Formula Z (F, J, G, and H). Following path 1, bromothiophene III is reacted with R²CH₂CH₂ZnCl or R²CH₂CH₂ZnBr, where R is as defined in Formula Z, in the presence of a palladium catalyst to afford compounds of Formula Z, where X is CH₂CH₂ (F). Alternatively, compounds of Formula J can be reduced by hydrogenation to give compounds of Formula Z, where X is

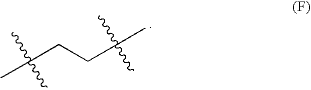

Following path 2 bromothiophene III is reacted with R²CHCHB(OH)₂, where R² is as defined in Formula Z, in the presence of a palladium catalyst to give compounds of Formula Z, where X is

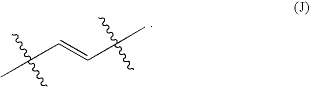

Following path 3 bromothiophene III is reacted with R²C(CH₂)B(OH)₂, where R² is as defined in Formula Z, in the presence of palladium to give compounds of Formula Z where X is

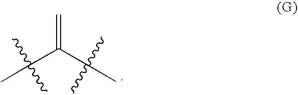

Compounds of Formula G are reacted with trimethylsufoxonium iodide under basic conditions to afford compounds of Formula Z, where X is

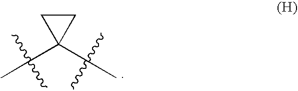

### EXAMPLES

### Example 1

### 3-(4-Amino-6-pyridin-2-ylethynyl-thieno[2,3-d]pyrimidin-2-yl)-benzonitrile

### Example 1

### Step A

### 3-(4-Amino-thieno[2,3-d]pyrimidin-2-yl)-benzonitrile

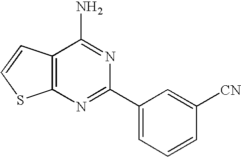

Solid potassium-tert-butoxide (1.1 g, 10.1 mmol) was added to a dioxane solution (20 mL) of 2-amino-thiophene-3-carbonitrile (5.0 g, 40.3 mmol) and 1,3-dicyanobenzene (7.2 g, 56.5 mmol). The resulting slurry was stirred vigorously at 130° C. for 15 minutes. The dark slurry was cooled to room temperature, diluted with THF, and dry packed onto silica gel. The material was the purified via column chromatography to give 10.2 g of the title compound.

### Example 1

### Step B

### 3-(4-Amino-6-bromo-thieno[2,3-d]pyrimidin-2-yl)-benzonitrile

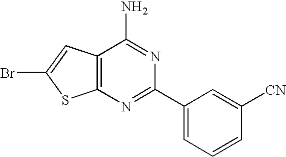

Solid NBS (1.6 g, 8.7 mmol) was added to a DMF solution (20 mL) of 3-(4-amino-thieno[2,3-d]pyrimidin-2-yl)-benzonitrile (2.0 g, 7.9 mmol). After 45 minutes water was added and the resulting precipitate was collected by filtration, washed with water, and dried in vacuo to give 2.4 g of the title compound.

### Example 1

### Step C

### 3-(4-Amino-6-pyridin-2-ylethynyl-thieno[2,3-d]pyrimidin-2-yl)-benzonitrile

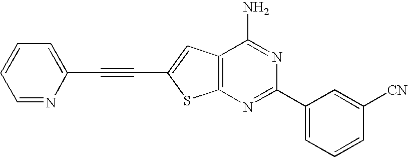

A THF solution of 3-(4-amino-6-bromo-thieno[2,3-d]pyrimidin-2-yl)-benzonitrile (150 mg, 0.45 mmol), 2-ethynyl-pyridine (51 mg, 0.50 mmol), CuI (17 mg, 0.09 mmol), Pd(dppf)Cl₂ (37 mg, 0.05 mmol), and Et₃N (0.31 mL, 2.25 mmol) was heated in the microwave at 100° C. for 30 min. The resulting mixture was diluted with THF and EtOAc and the organic layer was washed consecutively with 10% aqueous NH₄OH, water, and brine. The solution was dried (Na₂SO₄), dry packed onto silica gel, and purified via column chromatography to give 91 mg of the title compound. ¹H NMR (Acetone, 300 MHz): δ=8.73-8.81 (m, 2 H), 8.66 (br. s., 1 H), 7.95 (s, 1 H), 7.85-7.93 (m, 2 H), 7.65-7.78 (m, 2 H), 7.45 (dd, J=6.4, 4.9 Hz, 1 H), 7.32 ppm (br. s., 2 H); MS m/e 354 (M+H).

### Example 2

### 3-[4-Amino-6-(2-pyridin-2-yl-ethyl)-thieno[2,3-d]pyrimidin-2-yl]-benzonitrile

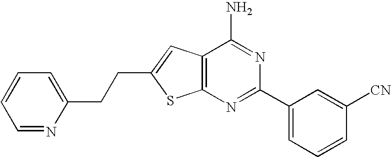

An EtOH solution (5 mL) of 3-(4-amino-6-pyridin-2-ylethynyl-thieno[2,3-d]pyrimidin-2-yl)-benzonitrile (35 mg, 0.10 mmol, prepared as described in Example 1) and 10% Pd/C (7 mg) was hydrogenated at 50 psi. After 16 h the solution was filtered through Celite and the filtrate was concentrated to give 30 mg of the title compound. ¹H NMR (Acetone, 300 MHz): δ=8.54-8.65 (m, 2 H), 8.41 (d, J=4.9 Hz, 1 H), 7.70 (d, J=7.5 Hz, 1 H), 7.48-7.61 (m, 2 H), 7.15 (t, J=4.0 Hz, 2 H), 7.07 (dd, J=7.5, 4.9 Hz, 1 H), 6.78 (br. s., 2 H), 3.20-3.33 (m, 2 H), 3.00-3.14 ppm (m, 2 H); MS m/e 358 (M+H).

### Example 3

### 6-(6-Chloro-pyridin-3-ylmethyl)-2-phenyl-thieno[2,3-d]pyrimidin-4-ylamine

### Example 3

### Step A

### 6-Bromo-2-phenyl-thieno[2,3-d]pyrimidin-4-ylamine

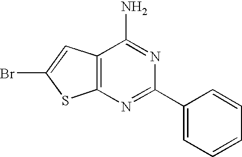

The title compound was prepared using benzonitrile in place of 1,3-dicyanobenzene as described in Example 1.

### Example 3

### Step B

### 6-(6-Chloro-pyridin-3-ylmethyl)-2-phenyl-thieno[2,3-d]pyrimidin-4-ylamine

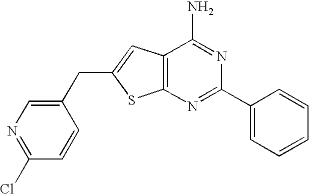

A 0.5 M THF solution of (6-chloro-3-pyridyl)methylzinc chloride (0.80 mL, 0.40 mmol) was added to a THF solution (1.6 mL) of 6-bromo-2-phenyl-thieno[2,3-d]pyrimidin-4-ylamine (50 mg, 0.16 mmol) and Pd(PPh₃)₄ (9 mg, 0.01 mmol) and the mixture was refluxed. After 3 h the mixture was diluted with EtOAc, washed with water then brine, dried (Na₂SO₄), concentrated and purified via column chromatography to give 21 mg of the title compound. ¹H NMR (Acetone, 300 MHz): δ=8.45 (dt, J=5.1, 2.4 Hz, 3 H), 7.77-7.89 (m, 1 H), 7.38-7.52 (m, 4 H), 7.24 (t, J=1.2 Hz, 1 H), 6.82 (br. s., 2 H), 4.34 ppm (s, 2 H); MS m/e 353 (M+H).

### Example 4

### 3-(4-Amino-6-pyrazin-2-ylmethyl-thieno[2,3-d]pyrimidin-2-yl)-benzonitrile

### Example 4

### Step A

### 2-But-3-enyl-pyrazine

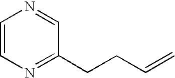

A 2.5 M hexanes solution of n-BuLi (18.0 mL, 45 mmol) was added to a −78° C. THF solution (60 mL) of t-BuOK (5.1 g, 45 mmol) and diisopropylamine (6.3 mL, 45 mmol). After 5 min at −78° C. the yellow mixture was warmed to −40° C. Neat methylpyrazine (2.7 mL, 30 mmol) was added and the mixture rapidly turned dark red. After 30 min at −40° C. the mixture was cooled to −78° C. and neat allyl bromide (7.6 mL, 90 mmol) was added. After 30 min at −78° C. water was added and the mixture was partially concentrated to remove volatile organics. The resulting mixture was extracted with dichloromethane and the combined organics were dried (Na₂SO₄), concentrated, and purified via column chromatography to give 2.2 g of 2-but-3-enyl-pyrazine.

### Example 4

### Step B

### 4-Pyrazin-2-yl-butane-1,2-diol

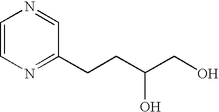

Osmium tetroxide (2.5 wt. % solution in t-BuOH, 4.0 mL, 0.32 mmol) was added to a 0° C. t-BuOH (30 mL)/water (30 mL) of 2-but-3-enyl-pyrazine (2.1 g, 15.8 mmol) and N-methyl morpholine N-oxide (2.0 g, 17.4 mmol) and the mixture was allowed to warm to rt overnight. TLC analysis indicated a low level of conversion, so an additional 8 mL of OsO₄ was added and the reaction mixture was stirred for 1 d. Conversion improved, but was still incomplete by TLC analysis; 0.5 equiv N-methyl morpholine N-oxide (925 mg) and 1.0 equiv pyridine (1.28 mL) were added, and the mixture was stirred for 2 h. A solution of 24 g Na₂SO₃ in 96 mL water was added, and the mixture was partially concentrated to remove volatile organics. The remaining aqueous solution was saturated with sodium chloride and was exhaustively extracted with ethyl acetate. The organic extracts were dried (Na₂SO₄), concentrated, and was purified by column chromatography to give 1.7 g of the title compound.

### Example 4

### Step C

### 3-Pyrazin-2-yl-propionaldehyde

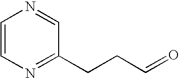

An aqueous solution of sodium periodate (0.65 M, 20 mL, 13 mmol, 1.3 equiv) was added to a suspension of silica gel (20 g) in dichloromethane (160 mL). A CH₂Cl₂ solution (10 mL) of 4-pyrazin-2-yl-butane-1,2-diol (1.7 g, 10.1 mmol) was then added. After 2 h the resulting white slurry was vacuum filtered and washed with CH₂Cl₂. The filtrate was dried (Na₂SO₄) and concentrated to give 1.1 g of the title compound that was used without further purification.

### Example 4

### Step D

### 2-Amino-5-pyrazin-2-ylmethyl-thiophene-3-carbonitrile

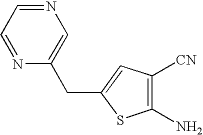

Solid elemental sulfur (257 mg, 8.0 mmol) was added to a 0° C. DMF solution (2 mL) of 3-pyrazin-2-yl-propionaldehyde (1.1 g, 8.0 mmol) and Et₃N (0.67 mL, 4.80 mmol). After 1 h, the solution was cooled to 0° C. and solid malononitrile (529 mg, 8.0 mmol) was added and stirred overnight. The mixture was partitioned between EtOAc and saturated aqueous sodium chloride, and the aqueous phase was extracted with EtOAc. The combined organic extracts were dried (Na₂SO₄), concentrated, and purified by column chromatography to give 555 mg of the title compound. ¹H NMR (CHLOROFORM-d, 300 MHz): δ (ppm) 8.44-8.59 (m, 3H), 6.54 (s, 1H), 4.73 (br. s., 2H), 4.12 (s, 2H)

### Example 4

### Step E

### 3-(4-Amino-6-pyrazin-2-ylmethyl-thieno[2,3-d]pyrimidin-2-yl)-benzonitrile

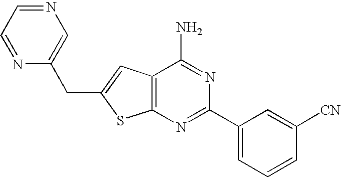

Solid t-BuOK (7 mg, 0.06 mmol) was added to a dioxane suspension (0.20 mL) of 2-amino-5-pyrazin-2-ylmethyl-thiophene-3-carbonitrile (70 mg, 0.32 mmol) and 1,3-dicyanobenzene (46 mg, 0.36 mmol) and the mixture was heated by microwave irradiation (150° C., 10 min, 300 W). The reaction mixture was diluted with dichloromethane and methanol, dry packed onto silica gel, and purified via column chromatography to give 83 mg of the title compound.

¹H NMR (DMSO-d₆, 300 MHz): δ=8.75 (s, 1 H), 8.54-8.68 (m, 4 H), 7.93 (d, J=7.5 Hz, 1 H), 7.70 (t, J=7.7 Hz, 1 H), 7.62 (br. s., 2 H), 7.37 (s, 1 H), 4.45 ppm (s, 2 H); MS m/e 345 (M+H).

### Example 5

### Step A

### 2-(1,1-Dimethyl-but-3-enyl)-pyridine

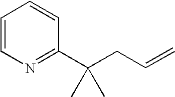

A 2.5 M hexanes solution of n-BuLi (18.0 mL, 45 mmol) was added to a −78° C. THF solution (60 mL) of t-BuOK (5.1 g, 45 mmol) and diisopropylamine (6.3 mL, 45 mmol). After 5 min at −78° C. the yellow mixture was warmed to −40° C. After 15 min, neat 2-isopropylpyridine (3.87 mL, 30 mmol) was added and the mixture rapidly turned dark red. After 30 min at −40° C. the mixture was cooled to −78° C. and neat allyl bromide (7.6 mL, 90 mmol) was added. After 30 min at −78° C. water was added and the mixture was partially concentrated to remove volatile organics. The resulting mixture was extracted with dichloromethane and the combined organics were dried (Na₂SO₄), concentrated, and purified via column chromatography to give 4.3 g of 2-(1,1-dimethyl-but-3-enyl)-pyridine.

### Example 5

### Step B

### 4-Methyl-4-pyridin-2-yl-pentane-1,2-diol

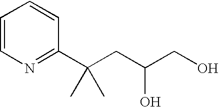

Osmium tetroxide (2.5 wt. % solution in t-BuOH, 13.4 mL, 1.1 mmol) was added to a 0° C. t-BuOH (40 mL)/water (40 mL) of 2-(1,1-dimethyl-but-3-enyl)-pyridine (3.5 g, 21.4 mmol) and N-methyl morpholine N-oxide (2.8 g, 23.6 mmol) and the mixture was allowed to warm to rt. After 3 h solid Na₂SO₃ (32 g) was added portionwise and the resulting suspension was stirred for 1 h. The mixture was partitioned between water and EtOAc and the aqueous phase was extracted with EtOAc. The combined organic extracts were dried (Na₂SO₄), concentrated, and was purified by column chromatography to give 3.9 g of the title compound.

### Example 5

### Step C

### 3-Methyl-3-pyridin-2-yl-butyraldehyde

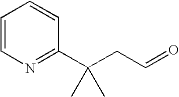

An aqueous solution of sodium periodate (0.65 M, 20 mL, 13 mmol) was added to a suspension of silica gel (20 g) in dichloromethane (160 mL). A CH₂Cl₂ solution (10 mL) solution of 4-methyl-4-pyridin-2-yl-pentane-1,2-diol (2.0 g, 10.0 mmol) was then added. After 1.5 h the resulting white slurry was vacuum filtered and washed with CH₂Cl₂. The filtrate was dried (Na₂SO₄) and concentrated to give 682 mg of the title compound.

### Example 5

### Step D

### 2-Amino-5-(1-methyl-1-pyridin-2-yl-ethyl)-thiophene-3-carbonitrile

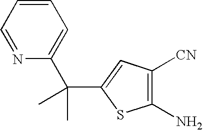

Solid elemental sulfur (110 mg, 3.4 mmol) was added to a 0° C. DMF solution (1 mL) of 3-methyl-3-pyridin-2-yl-butyraldehyde (671 mg, 4.1 mmol) and Et₃N (0.29 mL, 2.1 mmol). After 50 min, the solution was cooled to 0° C. and solid malononitrile (226 mg, 3.4 mmol) was added and stirred overnight. The mixture was partitioned between EtOAc and saturated aqueous sodium chloride, and the aqueous phase was extracted with EtOAc. The combined organic extracts were dried (Na₂SO₄), concentrated, and purified by column chromatography to give 430 mg of the title compound. ¹H NMR (CHLOROFORM-d, 300 MHz): ¹H NMR (CHLOROFORM-d, 300 MHz): δ (ppm) 8.57 (d, J=4.9 Hz, 1H), 7.61 (td, J=7.8, 2.1 Hz, 1H), 7.25-7.28 (m, 1H, obscured by CHCl₃ peak), 7.24 (dt, J=7.9, 1.1, 1H), 7.14 (ddd, J=7.5, 4.9, 1.1 Hz, 1H), 6.49 (s, 1H), 4.63 (br. s., 2H), 1.73 (s, 6H).

### Example 5

### Step E

### 3-[4-Amino-6-(1-methyl-1-pyridin-2-yl-ethyl)-thieno[2,3-d]pyrimidin-2-yl]-benzonitrile

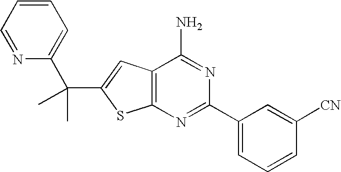

Solid t-BuOK (7 mg, 0.06 mmol) was added to a dioxane suspension (0.20 mL) of 2-amino-5-(1-methyl-1-pyridin-2-yl-ethyl)-thiophene-3-carbonitrile (75 mg, 0.31 mmol) and 1,3-dicyanobenzene (43 mg, 0.34 mmol) and the mixture was heated by microwave irradiation (150° C., 10 min, 300 W). The reaction mixture was diluted with dichloromethane and methanol, dry packed onto silica gel, and purified via column chromatography to give 83 mg of the title compound. ¹H NMR (DMSO-d₆, 300 MHz): δ=8.59-8.65 (m, 2 H), 8.57 (d, J=3.8 Hz, 1 H), 7.93 (d, J=7.9 Hz, 1 H), 7.76 (td, J=7.7, 1.9 Hz, 1 H), 7.69 (t, J=7.7 Hz, 1 H), 7.61 (br. s., 2 H), 7.51 (s, 1 H), 7.39 (d, J=7.9 Hz, 1 H), 7.26 (dd, J=7.5, 4.9 Hz, 1 H), 1.83 ppm (s, 6 H); MS m/e 372 (M+H).

### Example 6

### (+)-3-[4-Amino-6-(1-pyridin-2-yl-propyl)-thieno[2,3-d]pyrimidin-2-yl]-benzonitrile

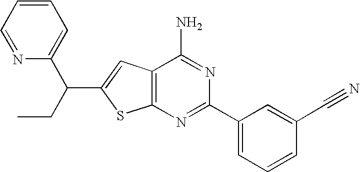

The title compound was prepared using 2-n-propylpyridine in place of 2-isopropylpyridine as described in Example 5. ¹H NMR (DMSO-d₆, 300 MHz): δ (ppm) 8.56-8.64 (m, 3H), 7.92 (d, J=7.5 Hz, 1H), 7.77 (td, 1H, J=7.6, 1.6 Hz), 7.69 (t, J=7.7 Hz, 1H), 7.60 (br s, 2H), 7.39-7.46 (m, 2H), 7.25-7.31 (m, 1H), 4.31 (t, J=7.7 Hz, 1H), 2.06-2.25 (m, 2H), 0.88 (t, J=7.2 Hz, 3H); MS m/e 372 (M+H).

### Example 7

### (±)-2-Phenyl-6-(1-pyridin-2-yl-propyl)-thieno[2,3-d]pyrimidin-4-ylamine hydrochloride

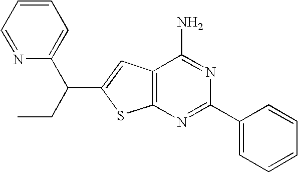

The title compound was prepared using 2-n-propylpyridine and benzonitrile in place of 2-isopropylpyridine and 1,3-dicyanobenzene, respectively as described in Example 6. ¹H NMR (DMSO-d₆, 300 MHz): δ (ppm) 8.75 (d, J=5.3 Hz, 1H), 8.31-8.37 (m, 2H), 8.18 (t, J=7.2 Hz, 1H), 7.77 (d, J=7.9 Hz, 1H), 7.61-7.67 (m, 2H), 7.51-7.59 (m, 3H), 4.64 (t, J=7.5 Hz, 1H), 2.14-2.29 (m, 2H), 0.91 (t, J=7.2 Hz, 3H); MS m/e 347 (M+H).

### Example 8

### 2-Phenyl-6-pyrazin-2-ylmethyl-thieno[2,3-d]pyrimidin-4-ylamine (14)

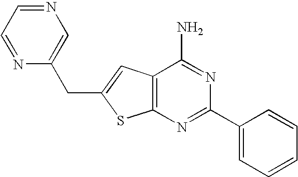

The title compound was prepared using benzonitrile in place of 1,3-dicyanobenzene as described in Example 5. ¹H NMR (DMSO-d₆, 300 MHz): δ (ppm) 8.75 (s, 1H), 8.64 (s, 1H), 8.58 (d, J=2.3 Hz, 1H), 8.29-8.37 (m, 2H), 7.42-7.54 (m, 5H), 7.34 (s, 1H), 4.43 (s, 2H); MS m/e 320 (M+H).

### Example 9

### 3-{4-Amino-6-[hydroxy-(2-methoxy-pyridin-3-yl)-methyl]-thieno[2,3-d]pyrimidin-2-yl}-benzonitrile

### Example 9

### Step A

### [2-(3-Cyano-phenyl)-thieno[2,3-d]pyrimidin-4-yl]-bis-carbamic acid tert-butyl ester

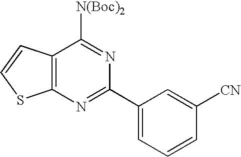

Solid DMAP (42 mg, 0.3 mmol) was added to a THF solution (17 mL) of 3-(4-Amino-thieno[2,3-d]pyrimidin-2-yl)-benzonitrile (850 mg, 3.4 mmol, an intermediate prepared in Example 1) and (Boc)₂O (1.8 g, 8.4 mmol). After 4 h the mixture was diluted with EtOAc and then washed consecutively with water and brine, dried (Na₂SO₄), concentrated and purified via column chromatography to give 1.2 g of the title compound.

### Example 9

### Step B

### {2-(3-Cyano-phenyl)-6-[hydroxy-(2-methoxy-pyridin-3-yl)-methyl]-thieno[2,3-d]pyrimidin-4-yl}-bis-carbamic acid tert-butyl ester

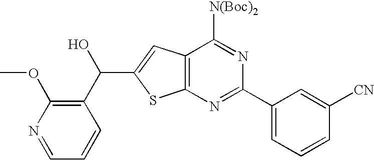

A 1.8 M THF solution of LDA (0.45 mL, 0.81 mmol) was added to a −78° C. THF solution (3 mL) of [2-(3-cyano-phenyl)-thieno[2,3-d]pyrimidin-4-yl]-bis-carbamic acid tert-butyl ester (310 mg, 0.68 mmol). After 3 min a THF solution (1 mL) of 2-methoxy-pyridine-3-carbaldehyde (0.09 mL, 0.81 mmol) was added and the reaction was warmed to room temperature. Saturated aqueous ammonium chloride was added and the crude reaction mixture was extracted with ethyl acetate. The combined extracts were dried (Na₂SO₄), concentrated and purified via column chromatography to give 130 mg of the title compound. ¹H NMR (300 MHz, CHLOROFORM-d) δ 8.70 (t, J=1.41 Hz, 1H), 8.63 (dt, J=1.48, 7.96 Hz, 1H), 8.09 (dd, J=1.79, 4.99 Hz, 1H), 7.62-7.70 (m, 2H), 7.46-7.54 (m, J=8.10 Hz, 1H), 7.45 (d, J=1.13 Hz, 1H), 7.33 (br. s., 1H), 6.89 (dd, J=4.90, 7.35 Hz, 1H), 6.18 (d, J=6.03 Hz, 1H), 3.95 (s, 3H), 3.36 (d, J=6.03 Hz, 1H), 1.49 (s, 9H); 490 (M+H).

### Example 9

### Step C

### 3-{4-Amino-6-[hydroxy-(2-methoxy-pyridin-3-yl)-methyl]-thieno[2,3-d]pyrimidin-2-yl}-benzonitrile

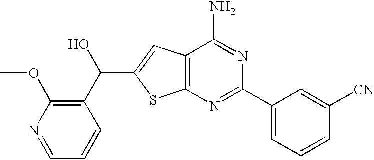

Neat trifluoroacetic acid (1 mL) was added dropwise to a CH₂Cl₂ solution (1 mL) of {2-(3-cyano-phenyl)-6-[hydroxy-(2-methoxy-pyridin-3-yl)-methyl]-thieno[2,3-d]pyrimidin-4-yl}-bis-carbamic acid tert-butyl ester (10 mg, 0.02 mmol). After 1 h The reaction was concentrated in vacuo and purified via HPLC. The compound dissolved in acetonitrile (5 mL) and stirred with 100 mg of Spectra/Gel® 1×8 strong-base anion, chloride form, ion-exchange resin and filtered to give 6 mg of the title compound as the HCl salt. ¹H NMR (300 MHz, DMSO-d₆) δ 8.53-8.69 (m, 2H), 8.14 (dd, J=1.88, 4.90 Hz, 1H), 7.91 (t, J=8.10 Hz, 2H), 7.69 (t, J=7.72 Hz, 1H), 7.61 (br. s., 2H), 7.34 (s, 1H), 7.08 (dd, J=4.90, 7.54 Hz, 1H), 6.51 (br. s., 1H), 6.10 (s, 1H), 3.92 (s, 3H); MS m/e 390 (M+H).

### Example 10

### 3-[4-Amino-6-(2-methoxy-pyridin-3-ylmethyl)-thieno[2,3-d]pyrimidin-2-yl]-benzonitrile

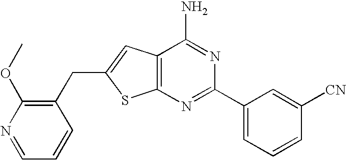

Neat triethylsilane (0.5 mL) was added to a CH₂Cl₂ (1 mL)/TFA (1 mL) solution of [2-(3-cyano-phenyl)-thieno[2,3-d]pyrimidin-4-yl]-bis-carbamic acid tert-butyl ester (30 mg, 0.06 mmol, an intermediate prepared in Example 9) and the mixture was heated to 70° C. After 5 h the mixture was cooled to room temperature, concentrated and purified via HPLC. The compound was dissolved in acetonitrile (5 mL) and stirred with 100 mg of Spectra/Gel 1×8 strong-base anion, chloride form ion-exchange resin, and then filtered to give 4 mg of the title compound as the HCl salt. ¹H NMR (300 MHz, MeOD) δ 8.56 (s, 1H), 8.48 (d, J=10.93 Hz, 1H), 8.08-8.18 (m, 1H), 7.98-8.08 (m, 1H), 7.80-7.88 (m, 1H), 7.74 (dd, J=1.79, 7.44 Hz, 1H), 7.39-7.49 (m, 1H), 7.03 (dd, J=5.09, 7.16 Hz, 1H), 4.30 (s, 2H), 4.01 (s, 3H). MS m/e 374 (M+H).

### Example 11

### 3-{4-Amino-6-[hydroxy-(6-methoxy-pyridin-2-yl)-methyl]-thieno[2,3-d]pyrimidin-2-yl}-benzonitrile

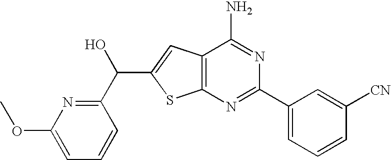

The title compound was prepared using 6-methoxy-pyridine-2-carbaldehyde in place of 2-methoxy-pyridine-3-carbaldehyde as described in Example 9. ¹H NMR (300 MHz, CHLOROFORM-d, MeOD) δ 8.63 (t, J=1.51 Hz, 1H), 8.55 (dt, J=1.46, 8.01 Hz, 1H), 7.78 (ddd, J=1.22, 1.37, 7.77 Hz, 1H), 7.55-7.71 (m, 2H), 7.42 (d, J=0.94 Hz, 1H), 7.07 d, J=7.35 Hz, 1H), 6.73 (d, J=8.29 Hz, 1H), 5.98 (s, 1H), 3.99 (s, 3H); MS m/e 390 (M+H).

### Example 12

### 3-{4-Amino-6-[hydroxy-(3,4,5,6-tetrahydro-2H-[1,2′]bipyridinyl-5′-yl)-methyl]-thieno[2,3-d]pyrimidin-2-yl}-benzonitrile

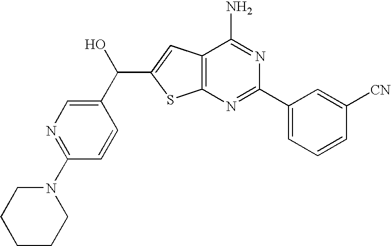

The title compound was prepared using 6-piperidin-1-ylnicotinaldehyde in place of 2-methoxy-pyridine-3-carbaldehyde as described in Example 9. ¹H NMR (300 MHz, MeOD) δ 8.46-8.76 (m, 2H), 7.91-8.11 (m, 2H), 7.83 (d, J=7.54 Hz, 1H), 7.56-7.74 (m, 1H), 7.31-7.48 (m, 2H), 6.09 (s, 1H), 3.57-3.84 (m, 4H), 1.80 (br. s., 6H); MS m/e 443 (M+H).

### Example 13

### 3-{4-Amino-6-[hydroxy-(2-morpholin-4-yl-pyridin-3-yl)-methyl]-thieno[2,3-d]pyrimidin-2-yl}-benzonitrile

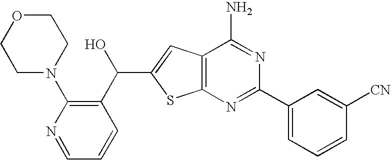

The title compound was prepared using 2-morpholin-4-ylpyridine-3-carboxaldehyde in place of 2-methoxy-pyridine-3-carbaldehyde as described in Example 9. ¹H NMR (300 MHz, MeOD) δ 8.49-8.63 (m, 2H), 8.42 (dd, J=1.70, 7.72 Hz, 1H), 8.34 (dd, J=1.88, 5.65 Hz, 1H), 7.83 (dt, J=1.37, 7.82 Hz, 1H), 7.59-7.70 (m, 1H), 7.45 (dd, J=5.65, 7.72 Hz, 1H), 7.40 (d, J=1.32 Hz, 1H), 6.35 (d, J=0.75 Hz, 1H), 3.84 (t, J=4.62 Hz, 4H), 3.42-3.55 (m, 2H), 3.29-3.42 (m, 2H); MS m/e 445 (M+H).

### Example 14

### 3-{4-Amino-6-[hydroxy-(2-pyrrolidin-1-yl-pyridin-3-yl)-methyl]-thieno[2,3-d]pyrimidin-2-yl}-benzonitrile

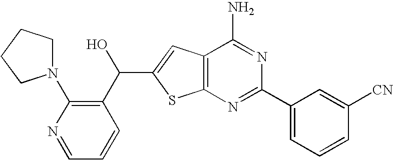

The title compound was prepared using 2-(1-pyrrolidinyl)nicotinaldehyde in place of 2-methoxy-pyridine-3-carbaldehyde as described in Example 9. ¹H NMR (300 MHz, DMSO-d₆) δ 8.52-8.71 (m, 2H), 8.15 (d, J=7.16 Hz, 1H), 8.06 (dd, J=1.51, 6.03 Hz, 1H), 7.89-7.99 (m, 1H), 7.63-7.77 (m, 2H), 7.35 (s, 1H), 7.05 (dd, J=6.40, 7.16 Hz, 1H), 6.40 (s, 1H), 3.67-3.87 (m, 4H), 1.84-2.07 (m, 4H); MS m/e 429 (M+H).

### Example 15

### 3-[4-Amino-6-(hydroxy-pyridin-2-yl-methyl)-thieno[2,3-d]pyrimidin-2-yl]-benzonitrile

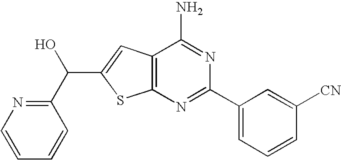

The title compound was prepared using pyridine-2-carbaldehyde in place of 2-methoxy-pyridine-3-carbaldehyde, as described in Example 9. ¹H NMR (300 MHz, MeOD) δ 8.76 (d, J=5.65 Hz, 1H), 8.57-8.72 (m, 2H), 8.47 (td, J=1.60, 7.86 Hz, 1H), 8.03 (d, J=8.10 Hz, 1H), 7.87-7.98 (m, 1H), 7.82 (dt, J=1.41, 7.72 Hz, 1H), 7.60-7.73 (m, 1H), 7.52 (d, J=0.94 Hz, 1H), 6.44 (s, 1H); MS m/e 360 (M+H).

### Example 16

### 2-Phenyl-6-pyridin-2-ylmethyl-thieno[2,3-d]pyrimidin-4-ylamine

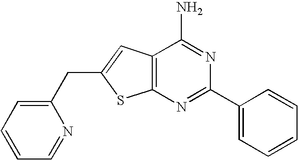

The title compound was prepared using benzonitrile in place of 1,3-dicyanobenzene as described in example 1, and pyridine-2-carbaldehyde in place of 2-methoxy-pyridine-3-carbaldehyde as described in Example 9. ¹H NMR (300 MHz, DMSO-d₆) δ 8.73 (d, J=4.52 Hz, 1H), 8.39 (td, J=1.70, 7.82 Hz, 1H), 8.13-8.31 (m, 2H), 7.72-7.94 (m, 2H), 7.44-7.58 (m, 3H), 7.39 (s, 1H), 4.62 (s, 2H); MS m/e 319 (M+H).

### Example 17

### 3-{4-Amino-6-[hydroxy-(3,4,5,6-tetrahydro-2H-[1,2′]bipyridinyl-3′-yl)-methyl]-thieno[2,3-d]pyrimidin-2-yl}-benzonitrile

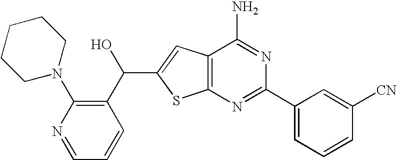

The title compound was prepared using 2-piperidin-1-ylpyridine-3-carbaldehyde in place of 2-methoxy-pyridine-3-carbaldehyde as described in Example 9. ¹H NMR (300 MHz, DMSO-d₆) δ 8.51-8.72 (m, 2H), 8.31 (dd, J=1.79, 5.37 Hz, 1H), 8.17 (d, J=7.91 Hz, 1H), 7.95 (dd, J=1.32, 7.72 Hz, 1H), 7.60-7.83 (m, 2H), 7.38 (s, 1H), 7.33 (dd, J=5.46, 7.35 Hz, 1H), 6.13 (s, 1H), 3.23-3.35 (m, 2H), 3.07-3.24 (m, 2H), 1.49-1.77 (m, 6H); MS m/e 443 (M+H).

### Example 18

### 3-{4-Amino-6-[(2-ethoxy-pyridin-3-yl)-hydroxy-methyl]-thieno[2,3-d]pyrimidin-2-yl}-benzonitrile

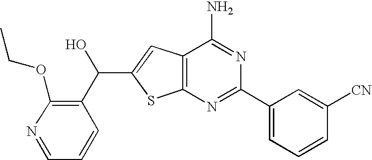

The title compound was prepared using 2-ethoxy-pyridine-3-carbaldehyde in place of 2-methoxy-pyridine-3-carbaldehyde as described in Example 9. ¹H NMR (300 MHz, MeOD) δ 8.52-8.72 (m, 2H), 8.08 (dd, J=1.88, 4.90 Hz, 1H), 7.98 (dd, J=1.88, 7.54 Hz, 1H), 7.80 (d, J=7.91 Hz, 1H), 7.64 (t, J=7.91 Hz, 1H), 7.29 (s, 1H), 7.03 (dd, J=5.09, 7.35 Hz, 1H), 6.24 (s, 1H), 4.39 (q, J=6.91 Hz, 2H), 1.35 (t, 3H); MS m/e 404 (M+H).

### Example 19

### 3-[4-Amino-6-(2-ethoxy-pyridin-3-ylmethyl)-thieno[2,3-d]pyrimidin-2-yl]-benzonitrile

### Example 19

### Step A

### {2-(3-Cyano-phenyl)-6-[(2-ethoxy-pyridin-3-yl)-hydroxy-methyl]-thieno[2,3-d]pyrimidin-4-yl}-bis-carbamic acid tert-butyl ester

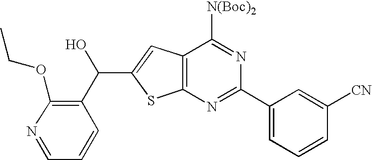

The title compound was prepared using 2-ethoxy-pyridine-3-carbaldehyde in place of 2-methoxy-pyridine-3-carbaldehyde as described in Example 9.

### Example 19

### Step B

### 3-[4-Amino-6-(2-ethoxy-pyridin-3-ylmethyl)-thieno[2,3-d]pyrimidin-2-yl]-benzonitrile

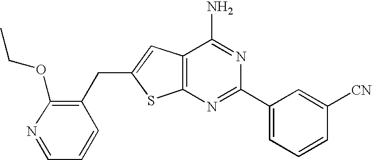

The title compound was prepared using {2-(3-cyano-phenyl)-6-[(2-ethoxy-pyridin-3-yl)-hydroxy-methyl]-thieno[2,3-d]pyrimidin-4-yl}-bis-carbamic acid tert-butyl ester in place of [2-(3-cyano-phenyl)-thieno[2,3-d]pyrimidin-4-yl]-bis-carbamic acid tert-butyl ester as described in Example 10. ¹H NMR (300 MHz, MeOD) δ 8.54-8.71 (m, 2H), 8.04 (dd, J=1.88, 5.27 Hz, 1H), 7.74-7.84 (m, 1H), 7.58-7.70 (m, 2H), 7.20 (s, 1H), 6.93 (dd, J=5.09, 7.35 Hz, 1H), 4.39 (q, J=7.03 Hz, 2H), 4.19 (s, 2H), 1.39 (t, J=6.97 Hz, 3H); MS m/e 388 (M+H).

### Example 20

### 3-[4-Amino-6-(3,4,5,6-tetrahydro-2H-[1,2′]bipyridinyl-3′-ylmethyl)-thieno[2,3-d]pyrimidin-2-yl]-benzonitrile

### Example 20

### Step A

### {2-(3-Cyano-phenyl)-6-[hydroxy-(3,4,5,6-tetrahydro-2H-[1,2′]bipyridinyl-3′-yl)-methyl]-thieno[2,3-d]pyrimidin-4-yl}-bis-carbamic acid tert-butyl ester

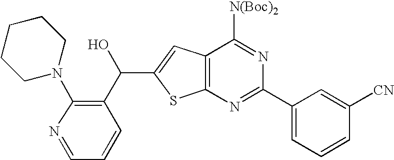

The title compound was prepared using 2-piperidin-1-ylpyridine-3-carboxaldehyde in place of 2-methoxy-pyridine-3-carbaldehyde as described in Example 9.

### Example 20

### Step B

### 3-[4-Amino-6-(3,4,5,6-tetrahydro-2H-[1,2′]bipyridinyl-3′-ylmethyl)-thieno[2,3-d]pyrimidin-2-yl]-benzonitrile

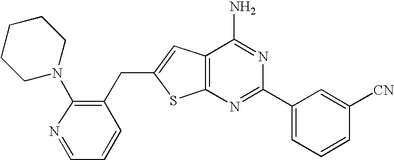

The title compound was prepared using {2-(3-cyano-phenyl)-6-[hydroxy-(3,4,5,6-tetrahydro-2H-[1,2′]bipyridinyl-3′-yl)-methyl]-thieno[2,3-d]pyrimidin-4-yl}-bis-carbamic acid tert-butyl ester in place of [2-(3-cyano-phenyl)-thieno[2,3-d]pyrimidin-4-yl]-bis-carbamic acid tert-butyl ester as described in Example 10. ¹H NMR (300 MHz, DMSO-d₆) δ 8.54-8.72 (m, 2H), 8.23 (dd, J=1.88, 5.27 Hz, 1H), 7.93 (d, J=7.91 Hz, 1H), 7.66-7.80 (m, 2H), 7.61 (br. s., 2H), 7.35 (s, 1H), 7.13 (dd, J=5.09, 7.35 Hz, 1H), 4.26 (s, 2H), 2.96-3.21 (m, 4H), 1.46-1.76 (m, 6H); MS m/e 427 (M+H).

### Example 21

### 3-{4-Amino-6-[(3-chloro-pyridin-4-yl)-hydroxy-methyl]-thieno[2,3-d]pyrimidin-2-yl}-benzonitrile

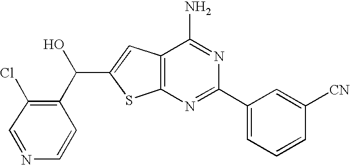

The title compound was prepared using 3-chloro-4-pyridinecarbaldehyde in place of 2-methoxy-pyridine-3-carbaldehyde as described in Example 9. ¹H NMR (300 MHz, DMSO-d₆) δ 8.51-8.78 (m, 4H), 7.93 (s, 1H), 7.51-7.82 (m, 4H), 7.34 (s, 1H), 6.20 (s, 1H); MS m/e 394 (M+H).

### Example 22

### 3-[4-Amino-6-(3-chloro-pyridin-4-ylmethyl)-thieno[2,3-d]pyrimidin-2-yl]-benzonitrile

### Example 22

### Step A

### [6-[(3-Chloro-pyridin-4-yl)-hydroxy-methyl]-2-(3-cyano-phenyl)-thieno[2,3-d]pyrimidin-4-yl]-bis-carbamic acid tert-butyl ester

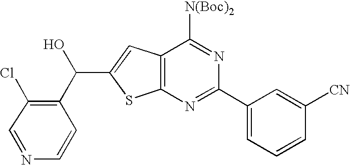

The title compound was prepared using 3-chloro-4-pyridinecarbaldehyde in place of 2-methoxy-pyridine-3-carbaldehyde as described in Example 9.

### Example 22

### Step B

### 3-[4-Amino-6-(3-chloro-pyridin-4-ylmethyl)-thieno[2,3-d]pyrimidin-2-yl]-benzonitrile

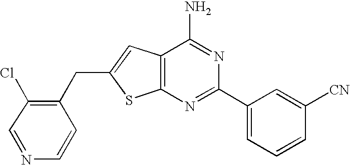

The title compound was prepared using [6-[(3-chloro-pyridin-4-yl)-hydroxy-methyl]-2-(3-cyano-phenyl)-thieno[2,3-d]pyrimidin-4-yl]-bis-carbamic acid tert-butyl ester in place of [2-(3-cyano-phenyl)-thieno[2,3-d]pyrimidin-4-yl]-bis-carbamic acid tert-butyl ester as described in Example 10. ¹H NMR (300 MHz, DMSO-d₆) δ 8.70 (s, 1H), 8.58-8.67 (m, 2H), 8.56 (d, J=4.90 Hz, 1H), 7.94 (d, J=7.54 Hz, 1H), 7.60-7.82 (m, 3H), 7.55 (d, J=4.90 Hz, 1H), 7.30 (s, 1H), 4.40 (s, 2H); MS m/e 378 (M+H).

### Example 23

### 3-(4-Amino-6-{hydroxy-[2-(2,2,2-trifluoro-ethoxy)-pyridin-3-yl]-methyl}-thieno[2,3-d]pyrimidin-2-yl)-benzonitrile

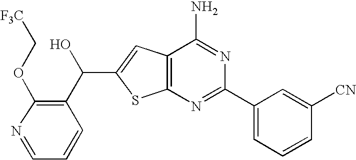

The title compound was prepared using 2-(2,2,2-trifluoro-ethoxy)-pyridine-3-carbaldehyde in place of 2-methoxy-pyridine-3-carbaldehyde as described in Example 9. ¹H NMR (300 MHz, CHLOROFORM-d) δ 8.67 (s, 1H), 8.59 (d, J=7.91 Hz, 1H), 8.11 (dd, J=1.70, 5.09 Hz, 1H), 8.02 (dd, J=1.88, 7.16 Hz, 1H), 7.72 (d, J=7.91 Hz, 1H), 7.53-7.63 (m, 1H), 7.18 (s, 1H), 7.10 (dd, J=5.09, 7.35 Hz, 1H), 6.30 (s, 1H), 4.80 (q, J=8.67 Hz, 2H); MS m/e 458 (M+H).

### Example 24

### 3-{4-Amino-6-[2-(2,2,2-trifluoro-ethoxy)-pyridin-3-ylmethyl]-thieno[2,3-d]pyrimidin-2-yl}-benzonitrile

### Example 24

### Step A

### (2-(3-Cyano-phenyl)-6-{hydroxy-[2-(2,2,2-trifluoro-ethoxy)-pyridin-3-yl]-methyl}-thieno[2,3-d]pyrimidin-4-yl)-bis-carbamic acid tert-butyl ester

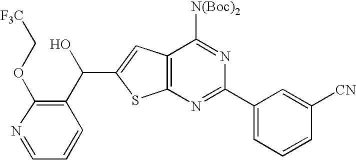

The title compound was prepared using 2-(2,2,2-trifluoro-ethoxy)-pyridine-3-carbaldehyde in place of 2-methoxy-pyridine-3-carbaldehyde as described in Example 9.

### Example 24

### Step B

### 3-{4-Amino-6-[2-(2,2,2-trifluoro-ethoxy)-pyridin-3-ylmethyl]-thieno[2,3-d]pyrimidin-2-yl}-benzonitrile

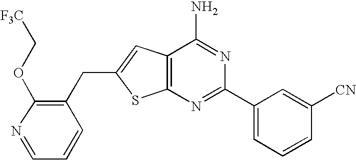

The title compound was prepared using (2-(3-cyano-phenyl)-6-{hydroxy-[2-(2,2,2-trifluoro-ethoxy)-pyridin-3-yl]-methyl}-thieno[2,3-d]pyrimidin-4-yl)-bis-carbamic acid tert-butyl ester in place of [2-(3-cyano-phenyl)-thieno[2,3-d]pyrimidin-4-yl]-bis-carbamic acid tert-butyl ester as described in Example 10. ¹H NMR (300 MHz, CHLOROFORM-d, MeOD) δ 8.61 (s, 1H), 8.53 (d, J=7.91 Hz, 1H), 8.02 (dd, J=1.88, 4.90 Hz, 1H), 7.61-7.74 (m, 1H), 7.48-7.61 (m, 2H), 7.04 (s, 1H), 6.96 (dd, J=5.09, 7.35 Hz, 1H), 4.76 (q, J=8.54 Hz, 2H), 4.17 (s, 2H); MS m/e 442 (M+H).

### Example 25

### 3-{4-Amino-6-[hydroxy-(2-isopropoxy-pyridin-3-yl)-methyl]-thieno[2,3-d]pyrimidin-2-yl}-benzonitrile

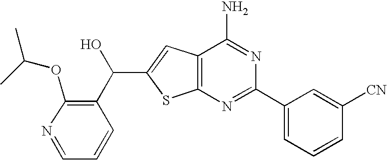

The title compound was prepared using 2-isopropoxy-pyridine-3-carbaldehyde in place of 2-methoxy-pyridine-3-carbaldehyde as described in Example 9. ¹H NMR (300 MHz, CHLOROFORM-d, MeOD) δ 8.67 (s, 1H), 8.58 (d, J=8.29 Hz, 1H), 8.09 (dd, J=1.88, 5.27 Hz, 1H), 7.88 (dd, J=1.51, 7.54 Hz, 1H), 7.67-7.78 (m, 1H), 7.50-7.64 (m, 1H), 7.14 (s, 1H), 6.96 (dd, J=4.90, 7.16 Hz, 1H), 6.21 (s, 1H), 5.32 (quin, J=6.12 Hz, 1H), 1.34 (d, J=6.40 Hz, 3H), 1.30 (d, J=6.03 Hz, 3H); MS m/e 418 (M+H).

### Example 26

### 3-[4-Amino-6-(2-isopropoxy-pyridin-3-ylmethyl)-thieno[2,3-d]pyrimidin-2-yl]-benzonitrile

### Example 26

### Step A

### {2-(3-Cyano-phenyl)-6-[hydroxy-(2-isopropoxy-pyridin-3-yl)-methyl]-thieno[2,3-d]pyrimidin-4-yl}-bis-carbamic acid tert-butyl ester

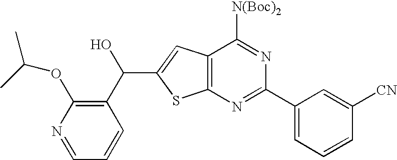

The title compound was prepared using 2-isopropoxy-pyridine-3-carbaldehyde in place of 2-methoxy-pyridine-3-carbaldehyde as described in Example 9.

### Example 26

### Step B

### 3-[4-Amino-6-(2-isopropoxy-pyridin-3-ylmethyl)-thieno[2,3-d]pyrimidin-2-yl]-benzonitrile

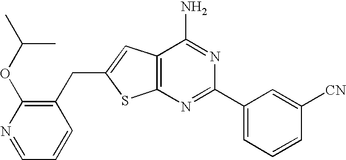

The title compound was prepared using {2-(3-cyano-phenyl)-6-[hydroxy-(2-isopropoxy-pyridin-3-yl)-methyl]-thieno[2,3-d]pyrimidin-4-yl}-bis-carbamic acid tert-butyl ester in place of [2-(3-cyano-phenyl)-thieno[2,3-d]pyrimidin-4-yl]-bis-carbamic acid tert-butyl ester as described in Example 10. ¹H NMR (300 MHz, CHLOROFORM-d) δ 8.74 (s, 1H), 8.65 (d, J=8.29 Hz, 1H), 8.08 (dd, J=1.88, 5.27 Hz, 1H), 7.70 (d, J=7.91 Hz, 1H), 7.51-7.60 (m, 1H), 7.46 (dd, J=1.88, 7.16 Hz, 1H), 6.73-6.91 (m, 2H), 5.37 (quin, J=6.12 Hz, 1H), 5.27 (br. s., 2H), 4.14 (s, 2H), 1.36 (d, J=6.03 Hz, 6H); MS m/e 402 (M+H).

### Example 27

### 3-[4-Amino-6-(2-morpholin-4-yl-pyridin-3-ylmethyl)-thieno[2,3-d]pyrimidin-2-yl]-benzonitrile

### Example 27

### Step A

### {2-(3-Cyano-phenyl)-6-[hydroxy-(2-morpholin-4-yl-pyridin-3-y)-methyl]-thieno[2,3-d]pyrimidin-4-yl}-bis-carbamic acid tert-butyl ester

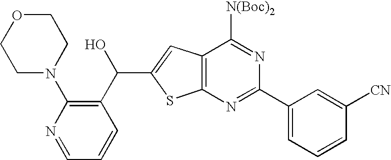

The title compound was prepared using 2-morpholin-4-ylpyridine-3-carboxaldehyde in place of 2-methoxy-pyridine-3-carbaldehyde as described in Example 9.

### Example 27

### Step B

### 3-[4-Amino-6-(2-morpholin-4-yl-pyridin-3-ylmethyl)-thieno[2,3-d]pyrimidin-2-yl]-benzonitrile

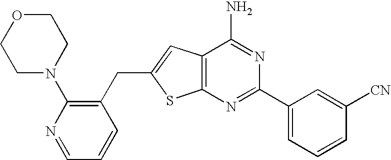

The title compound was prepared using {2-(3-cyano-phenyl)-6-[hydroxy-(2-morpholin-4-yl-pyridin-3-yl)-methyl]-thieno[2,3-d]pyrimidin-4-yl}-bis-carbamic acid tert-butyl ester in place of [2-(3-cyano-phenyl)-thieno[2,3-d]pyrimidin-4-yl]-bis-carbamic acid tert-butyl ester as described in Example 10. ¹H NMR (300 MHz, DMSO-d₆) δ 8.53-8.69 (m, 2H), 8.28 (d, J=5.09 Hz, 1H), 7.94 (d, J=6.59 Hz, 1H), 7.62-7.86 (m, 3H), 7.38 (s, 1H), 7.14-7.27 (m, 1H), 4.32 (s, 2H), 3.64-3.81 (m, 4H), 3.10-3.25 (m, 4H); MS m/e 429 (M+H).

### Example 28

### 3-[4-Amino-6-(hydroxy-pyridin-4-yl-methyl)-thieno[2,3-d]pyrimidin-2-yl]-benzonitrile

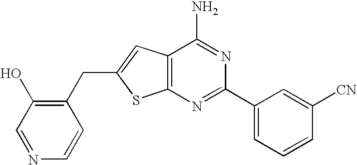

The title compound was prepared using pyridine-4-carboxaldehyde in place of 2-methoxy-pyridine-3-carbaldehyde as described in Example 9. ¹H NMR (300 MHz, MeOD) δ 8.87 (d, J=6.78 Hz, 2H), 8.56-8.74 (m, 2H), 8.24 (d, J=6.59 Hz, 2H), 7.76-7.88 (m, 1H), 7.59-7.72 (m, 1H), 7.51 (s, 1H), 6.39 (s, 1H); MS m/e 360 (M+H).

### Example 29

### 3-{4-Amino-6-[(2-fluoro-pyridin-3-yl)-hydroxy-methyl]-thieno[2,3-d]pyrimidin-2-yl}-benzonitrile

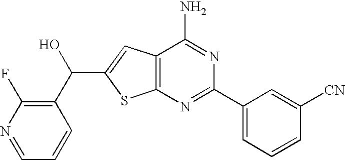

The title compound was prepared using 2-fluoropyridine-3-carbaldehyde in place of 2-methoxy-pyridine-3-carbaldehyde as described in Example 9. ¹H NMR (400 MHz, MeOD) δ 8.63-8.69 (m, 1H), 8.57-8.62 (m, 1H), 8.16-8.29 (m, 2H), 7.83 (dt, J=1.44, 7.64 Hz, 1H), 7.67 (t, J=7.95 Hz, 1H), 7.43 (ddd, J=1.59, 5.14, 7.21 Hz, 1H), 7.36 (s, 1H), 6.30 (s, 1H); MS m/e 378 (M+H).

### Example 30

### 3-{4-Amino-6-[(2-chloro-pyridin-3-yl)-hydroxy-methyl]-thieno[2,3-d]pyrimidin-2-yl}-benzonitrile

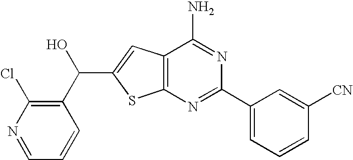

The title compound was prepared using 2-chloropyridine-3-carbaldehyde in place of 2-methoxy-pyridine-3-carbaldehyde as described in Example 9. ¹H NMR (300 MHz, MeOD) δ 8.51-8.70 (m, 2H), 8.37 (dd, J=1.88, 4.90 Hz, 1H), 8.24 (dd, J=1.88, 7.72 Hz, 1H), 7.78-7.91 (m, 1H), 7.62-7.73 (m, 1H), 7.52 (dd, J=4.71, 7.72 Hz, 1H), 7.36 (d, J=1.13 Hz, 1H), 6.36 (s, 1H); MS m/e 394 (M+H).

### Example 31

### 3-[4-Amino-6-(hydroxy-pyridin-3-yl-methyl)-thieno[2,3-d]pyrimidin-2-yl]-benzonitrile

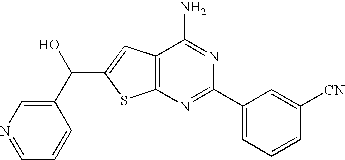

The title compound was prepared using pyridine-3-carbaldehyde in place of 2-methoxy-pyridine-3-carbaldehyde as described in Example 9. ¹H NMR (300 MHz, MeOD) δ 9.10 (d, J=1.13 Hz, 1H), 8.90 (d, J=5.65 Hz, 1H), 8.74-8.84 (m, 1H), 8.63 (t, J=1.51 Hz, 1H), 8.57 (ddd, J=1.32, 1.46, 8.15 Hz, 1H), 8.18 (dd, J=5.84, 8.10 Hz, 1H), 7.97 (dt, J=1.34, 7.86 Hz, 1H), 7.70-7.84 (m, 1H), 7.62 (d, J=0.94 Hz, 1H), 6.47 (s, 1H); MS m/e 360 (M+H).

### Example 32

### 3-(4-Amino-6-pyridin-3-ylmethyl-thieno[2,3-d]pyrimidin-2-yl)-benzonitrile

### Example 32

### Step A

### [2-(3-Cyano-phenyl)-6-(hydroxy-pyridin-3-yl-methyl)-thieno[2,3-d]pyrimidin-4-yl]-bis-carbamic acid tert-butyl ester

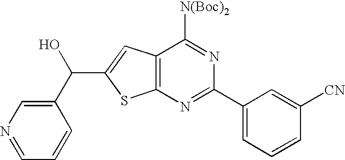

The title compound was prepared using pyridine-3-carbaldehyde in place of 2-methoxy-pyridine-3-carbaldehyde as described in Example 9.

### Example 32

### Step B

### 3-(4-Amino-6-pyridin-3-ylmethyl-thieno[2,3-d]pyrimidin-2-yl)-benzonitrile

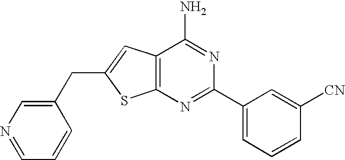

The title compound was prepared using [2-(3-cyano-phenyl)-6-(hydroxy-pyridin-3-yl-methyl)-thieno[2,3-d]pyrimidin-4-yl]-bis-carbamic acid tert-butyl ester in place of [2-(3-cyano-phenyl)-thieno[2,3-d]pyrimidin-4-yl]-bis-carbamic acid tert-butyl ester as described in Example 10. ¹H NMR (400 MHz, MeOD) δ 8.79 (d, J=1.96 Hz, 1H), 8.71 (dd, J=1.22, 5.38 Hz, 1H), 8.64-8.69 (m, 1H), 8.61 (dt, J=1.47, 8.07 Hz, 1H), 8.39 (d, J=7.83 Hz, 1H), 7.92 (dd, J=5.50, 7.95 Hz, 1H), 7.78 (ddd, J=1.35, 1.47, 7.70 Hz, 1H), 7.59-7.66 (m, 1H), 7.32 (s, 1H), 4.49 (s, 2H); MS m/e 344 (M+H).

### Example 33

### 3-{4-Amino-6-[hydroxy-(3-methoxy-pyridin-2-yl)-methyl]-thieno[2,3-d]pyrimidin-2-yl}-benzonitrile

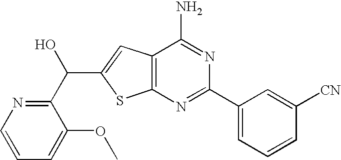

The title compound was prepared using 3-methoxy-pyridine-2-carbaldehyde in place of 2-methoxy-pyridine-3-carbaldehyde as described in Example 9. ¹H NMR (300 MHz, MeOD) δ 8.65-8.71 (m, 1H), 8.58-8.65 (m, 1H), 8.38 (dd, J=1.04, 5.56 Hz, 1H), 8.21 (d, J=8.85 Hz, 1H), 7.96 (dd, J=5.65, 8.67 Hz, 1H), 7.77-7.85 (m, 1H), 7.60-7.69 (m, 1H), 7.51 (d, J=0.75 Hz, 1H), 6.57 (s, 1H), 4.11 (s, 3H); MS m/e 390 (M+H).

### Example 34

### 3-[4-Amino-6-(3-methoxy-pyridin-2-ylmethyl)-thieno[2,3-d]pyrimidin-2-yl]-benzonitrile

### Example 34

### Step A

### {2-(3-Cyano-phenyl)-6-[hydroxy-(3-methoxy-pyridin-2-yl)-methyl]-thieno[2,3-d]pyrimidin-4-yl}-bis-carbamic acid tert-butyl ester

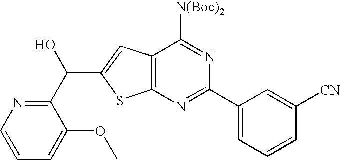

The title compound was prepared using 3-methoxy-pyridine-2-carbaldehyde in place of 2-methoxy-pyridine-3-carbaldehyde as described in Example 9.

### Example 34

### Step B

### 3-[4-Amino-6-(3-methoxy-pyridin-2-ylmethyl)-thieno[2,3-d]pyrimidin-2-yl]-benzonitril

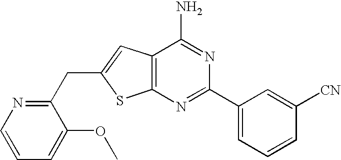

The title compound was prepared using {2-(3-cyano-phenyl)-6-[hydroxy-(3-methoxy-pyridin-2-yl)-methyl]-thieno[2,3-d]pyrimidin-4-yl}-bis-carbamic acid tert-butyl ester in place of [2-(3-cyano-phenyl)-thieno[2,3-d]pyrimidin-4-yl]-bis-carbamic acid tert-butyl ester as described in Example 10. ¹H NMR (300 MHz, MeOD) δ 8.65 (d, J=1.13 Hz, 1H), 8.61 (d, J=7.91 Hz, 1H), 8.27 (d, J=5.46 Hz, 1H), 8.00 (d, J=8.48 Hz, 1H), 7.70-7.86 (m, 2H), 7.59-7.70 (m, 1H), 7.30 (s, 1H), 4.57 (s, 2H), 4.08 (s, 3H); MS m/e 374 (M+H).

### Example 35

### 3-[4-Amino-6-(2-chloro-pyridin-3-ylmethyl)-thieno[2,3-d]pyrimidin-2-yl]-benzonitrile

### Example 35

### Step A

### [6-[(2-Chloro-pyridin-3-yl)-hydroxy-methyl]-2-(3-cyano-phenyl)-thieno[2,3-d]pyrimidin-4-yl]-bis-carbamic acid tert-butyl ester

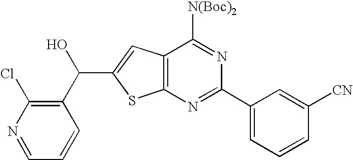

The title compound was prepared using 2-chloropyridine-3-carbaldehyde in place of 2-methoxy-pyridine-3-carbaldehyde as described in Example 9.

### Example 35

### Step B

### 3-[4-Amino-6-(2-chloro-pyridin-3-ylmethyl)-thieno[2,3-d]pyrimidin-2-yl]-benzonitrile

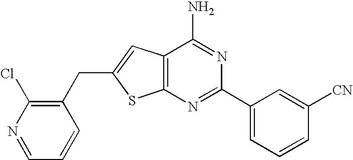

The title compound was prepared using [6-[(2-chloro-pyridin-3-yl)-hydroxy-methyl]-2-(3-cyano-phenyl)-thieno[2,3-d]pyrimidin-4-yl]-bis-carbamic acid tert-butyl ester in place of [2-(3-cyano-phenyl)-thieno[2,3-d]pyrimidin-4-yl]-bis-carbamic acid tert-butyl ester as described in Example 10. ¹H NMR (300 MHz, MeOD) δ 8.64-8.69 (m, 1H), 8.57-8.64 (m, 1H), 8.33 (dd, J=1.88, 4.71 Hz, 1H), 7.90 (dd, J=1.88, 7.54 Hz, 1H), 7.77-7.85 (m, 1H), 7.60-7.70 (m, 1H), 7.42 (dd, J=4.80, 7.63 Hz, 1H), 7.18-7.27 (m, 1H), 4.40 (s, 2H); MS m/e 378 (M+H).

### Example 36

### 3-{4-Amino-6-[hydroxy-(6-methoxy-pyridin-3-yl)-methyl]-thieno[2,3-d]pyrimidin-2-yl}-benzonitrile

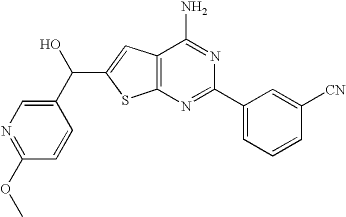

The title compound was prepared using 6-methoxy-pyridine-3-carbaldehyde in place of 2-methoxy-pyridine-3-carbaldehyde as described in Example 9. ¹H NMR (300 MHz, DMSO-d₆) δ 8.50-8.73 (m, 2H), 8.27 (d, J=2.45 Hz, 1H), 7.85-8.03 (m, 1H), 7.59-7.81 (m, 2H), 7.29 (d, J=1.13 Hz, 1H), 6.86 (d, J=8.85 Hz, 1H), 5.99 (s, 1H), 3.87 (s, 3H); MS m/e 390 (M+H).

### Example 37

### 3-[4-Amino-6-(6-methoxy-pyridin-3-ylmethyl)-thieno[2,3-d]pyrimidin-2-yl]-benzonitrile

### Example 37

### Step A

### {2-(3-Cyano-phenyl)-6-[hydroxy-(6-methoxy-pyridin-3-yl)-methyl]-thieno[2,3-d]pyrimidin-4-yl}-bis-carbamic acid tert-butyl ester

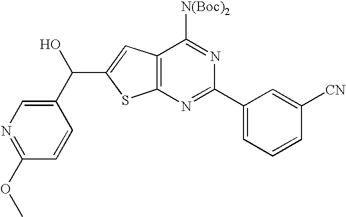

The title compound was prepared using 6-methoxy-pyridine-3-carbaldehyde in place of 2-methoxy-pyridine-3-carbaldehyde, as described in Example 9.

### Example 37

### Step B

### 3-[4-Amino-6-(6-methoxy-pyridin-3-ylmethyl)-thieno[2,3-d]pyrimidin-2-yl]-benzonitril

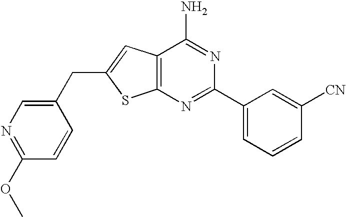

The title compound was prepared using {2-(3-cyano-phenyl)-6-[hydroxy-(6-methoxy-pyridin-3-yl)-methyl]-thieno[2,3-d]pyrimidin-4-yl}-bis-carbamic acid tert-butyl ester in place of [2-(3-cyano-phenyl)-thieno[2,3-d]pyrimidin-4-yl]-bis-carbamic acid tert-butyl ester as described in Example 10. ¹H NMR (300 MHz, DMSO-d₆) δ 8.53-8.69 (m, 2H), 8.17 (d, J=2.64 Hz, 1H), 7.87-8.00 (m, 1H), 7.60-7.76 (m, 2H), 7.28 (s, 1H), 6.83 (d, J=8.48 Hz, 1H), 4.19 (s, 2H), 3.85 (s, 3H); MS m/e 374 (M+H).

### Example 38

### 3-[4-Amino-6-(3-fluoro-pyridin-2-ylmethyl)-thieno[2,3-d]pyrimidin-2-yl]-benzonitrile

### Example 38

### Step A

### {2-(3-Cyano-phenyl)-6-[(3-fluoro-pyridin-2-yl)-hydroxy-methyl]-thieno[2,3-d]pyrimidin-4-yl}-bis-carbamic acid tert-butyl ester

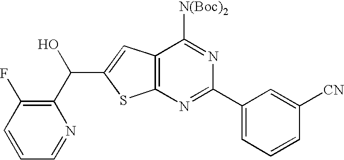

The title compound was prepared using 3-fluoro-2-formylpyridine in place of 2-methoxy-pyridine-3-carbaldehyde as described in Example 9.

### Example 38

### Step B

### 3-[4-Amino-6-(3-fluoro-pyridin-2-ylmethyl)-thieno[2,3-d]pyrimidin-2-yl]-benzonitrile

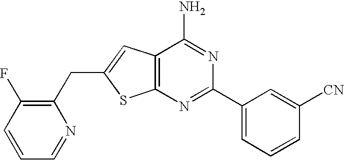

The title compound was prepared using {2-(3-cyano-phenyl)-6-[(3-fluoro-pyridin-2-yl)-hydroxy-methyl]-thieno[2,3-d]pyrimidin-4-yl}-bis-carbamic acid tert-butyl ester in place of [2-(3-cyano-phenyl)-thieno[2,3-d]pyrimidin-4-yl]-bis-carbamic acid tert-butyl ester as described in Example 10. ¹H NMR (300 MHz, DMSO-d₆) δ 8.53-8.68 (m, 2H), 8.44 (dt, J=1.44, 4.66 Hz, 1H), 7.95 (ddd, J=1.22, 1.37, 7.77 Hz, 1H), 7.64-7.86 (m, 2H), 7.33-7.51 (m, 2H), 4.43 (d, 2H); MS m/e 362 (M+H).

### Example 39

### 3-[4-Amino-6-(2-methoxy-pyridine-3-carbonyl)-thieno[2,3-d]pyrimidin-2-yl]-benzonitrile

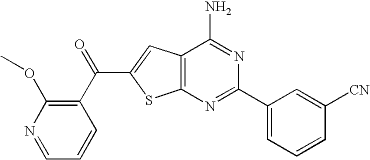

Solid Dess-Martin reagent (16 mg, 0.04 mmol) was added to a CH₂Cl₂ solution (2 mL) of {2-(3-cyano-phenyl)-6-[hydroxy-(2-methoxy-pyridin-3-yl)-methyl]-thieno[2,3-d]pyrimidin-4-yl}-bis-carbamic acid tert-butyl ester (20 mg, 0.04 mmol, prepared as an intermediate in Example 9). After 2 h the reaction mixture was concentrated in vacuo and purified via column chromatography to give the corresponding ketone, that was then dissolved in CH₂Cl₂(1 mL)/TFA (1 mL) and stirred. After 1 hour the reaction mixture was concentrated in vacuo and purified via HPLC to give 5 mg of the title compound as the TFA salt. ¹H NMR (300 MHz, MeOD) δ 8.55 (t, J=1.51 Hz, 1H), 8.48 (dt, J=1.51, 8.10 Hz, 1H), 8.32 (dd, J=1.88, 5.09 Hz, 1H), 8.07 (s, 1H), 7.94 (dt, J=1.27, 7.82 Hz, 1H), 7.85 (dd, J=1.88, 7.35 Hz, 1H), 7.67-7.77 (m, 1H), 7.08 (dd, J=5.09, 7.35 Hz, 1H), 3.87 (s, 3H); MS m/e 388 (M+H).

### Example 40

### 6-(6-Chloro-pyridin-3-ylmethyl)-2-(3-fluoro-phenyl)-thieno[2,3-d]pyrimidin-4-ylamine

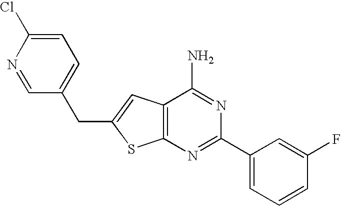

The title compound was prepared using 3-fluoro-benzonitrile in place of benzonitrile as described in Example 3. ¹H NMR (300 MHz, CDCl₃) δ=8.38 (d, J=2.3 Hz, 1 H), 8.21 (d, J=7.9 Hz, 1 H), 8.12 (dt, J=2.1, 10.5 Hz, 1 H), 7.59 (dd, J=2.4, 8.1 Hz, 1 H), 7.42 (td, J=5.8, 8.0 Hz, 1 H), 7.34 (d, J=7.9 Hz, 1 H), 7.14 (td, J=1.9, 8.3 Hz, 1 H), 6.78 (s, 1 H), 5.21 (br. s., 2 H), 4.22 (s, 2 H); MS m/e 371/373 (M+H).

### Example 41

### 3-{4-Amino-6-[(3-chloro-pyridin-2-yl)-hydroxy-methyl]-thieno[2,3-d]pyrimidin-2-yl}-benzonitrile

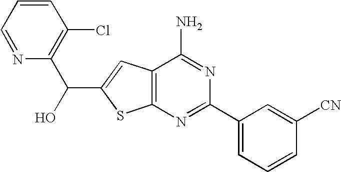

The title compound was prepared using 3-chloro-pyridine-2-carbaldehyde in place of 2-methoxy-pyridine-3-carbaldehyde, as described in Example 9. ¹H NMR (300 MHz, Acetone-d₆) δ=8.67-8.74 (m, 2 H), 8.64 (d, J=3.4 Hz, 1 H), 7.92-8.01 (m, 1 H), 7.83 (d, J=7.9 Hz, 1 H), 7.67 (t, J=8.1 Hz, 1 H), 7.49 (dd, J=4.7, 8.1 Hz, 1 H), 7.41 (s, 1 H), 7.03 (br. s., 2 H), 6.38 (s, 1 H); MS m/e 394/396 (M+H).

### Example 42

### 3-[4-Amino-6-(3-chloro-pyridin-2-ylmethyl)-thieno[2,3-d]pyrimidin-2-yl]-benzonitrile

### Example 42

### Step A

### [6-[(3-Chloro-pyridin-2-yl)-hydroxy-methyl]-2-(3-cyano-phenyl)-thieno[2,3-d]pyrimidin-4-yl]-bis-carbamic acid tert-butyl ester

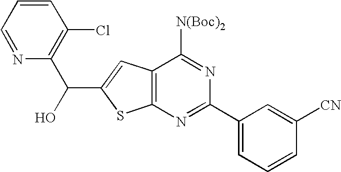

The title compound was prepared using 3-chloro-pyridine-2-carbaldehyde in place of 2-methoxy-pyridine-3-carbaldehyde, as described in Example 9.

### Example 42

### Step B

### 3-[4-Amino-6-(3-chloro-pyridin-2-ylmethyl)-thieno[2,3-d]pyrimidin-2-yl]-benzonitrile

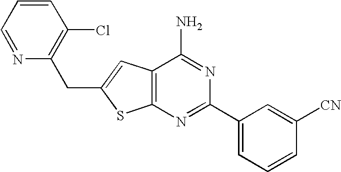

The title compound was prepared using [6-[(3-chloro-pyridin-2-yl)-hydroxy-methyl]-2-(3-cyano-phenyl)-thieno[2,3-d]pyrimidin-4-yl]-bis-carbamic acid tert-butyl ester in place of [2-(3-cyano-phenyl)-thieno[2,3-d]pyrimidin-4-yl]-bis-carbamic acid tert-butyl ester as described in Example 10. ¹H NMR (300 MHz, Acetone-d₆) δ=8.72 (s, 1 H), 8.69 (s, 1 H), 8.53 (d, J=4.5 Hz, 1 H), 7.88 (d, J=7.9 Hz, 1 H), 7.83 (d, J=7.5 Hz, 1 H), 7.67 (t, J=7.7 Hz, 1 H), 7.35-7.40 (m, 1 H), 7.34 (s, 1 H), 6.98 (br. s., 2 H), 4.55 (s, 2 H); MS m/e 378/380 (M+H).

### Example 43

### 3-{4-Amino-6-[(3-bromo-pyridin-2-yl)-hydroxy-methyl]-thieno[2,3-d]pyrimidin-2-yl}-benzonitrile

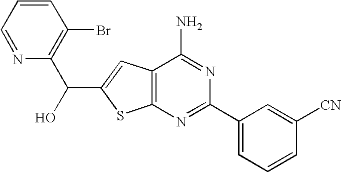

The title compound was prepared using 3-bromo-pyridine-2-carbaldehyde in place of 2-methoxy-pyridine-3-carbaldehyde, as described in Example 9. ¹H NMR (400 MHz, Acetone-d₆) δ=8.68-8.72 (m, 3 H), 8.13 (dd, J=1.2, 8.1 Hz, 1 H), 7.79-7.87 (m, 1 H), 7.63-7.73 (m, 1 H), 7.37-7.46 (m, 2 H), 7.01 (br. s, 2 H), 6.35 (d, J=7.3 Hz, 1 H), 5.52 (d, J=7.8 Hz, 1 H); MS m/e 438/440 (M+H).

### Example 44

### 3-[4-Amino-6-(3-bromo-pyridin-2-ylmethyl)-thieno[2,3-d]pyrimidin-2-yl]-benzonitrile

### Example 44

### Step A

### [6-[(3-Bromo-pyridin-2-yl)-hydroxy-methyl]-2-(3-cyano-phenyl)-thieno[2,3-d]pyrimidin-4-yl]-bis-carbamic acid tert-butyl ester

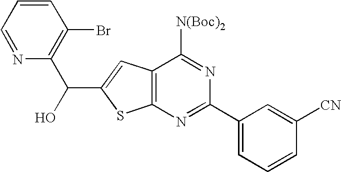

The title compound was prepared using 3-bromo-pyridine-2-carbaldehyde in place of 2-methoxy-pyridine-3-carbaldehyde, as described in Example 9.

### Example 44

### Step B

### 3-[4-Amino-6-(3-bromo-pyridin-2-ylmethyl)-thieno[2,3-d]pyrimidin-2-yl]-benzonitrile

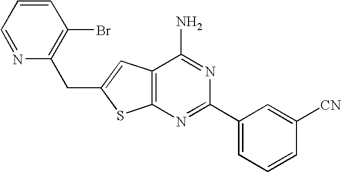

The title compound was prepared using [6-[(3-bromo-pyridin-2-yl)-hydroxy-methyl]-2-(3-cyano-phenyl)-thieno[2,3-d]pyrimidin-4-yl]-bis-carbamic acid tert-butyl ester in place of [2-(3-cyano-phenyl)-thieno[2,3-d]pyrimidin-4-yl]-bis-carbamic acid tert-butyl ester as described in Example 10. ¹H NMR (300 MHz, Acetone-d₆) δ=8.65-8.76 (m, 2 H), 8.53-8.62 (m, 1 H), 8.06 (d, J=8.3 Hz, 1 H), 7.83 (d, J=7.9 Hz, 1 H), 7.68 (t, J=8.1 Hz, 1 H), 7.35 (s, 1 H), 7.28 (dd, J=4.7, 8.1 Hz, 1 H), 6.98 (br. s., 2 H), 4.58 (s, 2 H); MS m/e 422/424 (M+H).

Biological Assays and Activity

Ligand Binding Assay for Adenosine A2a Receptor

Ligand binding assay of adenosine A2a receptor was performed using plasma membrane of HEK293 cells containing human A2a adenosine receptor (PerkinElmer, RB-HA2a) and radioligand [³H]CGS21680 (PerkinElmer, NET1021). Assay was set up in 96-well polypropylene plate in total volume of 200 μL by sequentially adding 20 μL 1:20 diluted membrane, 130 μL assay buffer (50 mM Tris.HCl, pH 7.4 10 mM MgCl₂, 1 mM EDTA) containing [³H] CGS21680, 50 μL diluted compound (4×) or vehicle control in assay buffer. Nonspecific binding was determined by 80 mM NECA. Reaction was carried out at room temperature for 2 hours before filtering through 96-well GF/C filter plate pre-soaked in 50 mM Tris.HCl, pH 7.4 containing 0.3% polyethylenimine. Plates were then washed 5 times with cold 50 mM Tris.HCl, pH 7.4, dried and sealed at the bottom. Microscintillation fluid 30 μL was added to each well and the top sealed. Plates were counted on Packard Topcount for [³H]. Data was analyzed in Microsoft Excel and GraphPad Prism programs. (Varani, K.; Gessi, S.; Dalpiaz, A.; Borea, P. A. British Journal of Pharmacology, 1996, 117, 1693)

Adenosine A2a Receptor Functional Assay (A2AGAL2)

To initiate the functional assay, cryopreserved CHO-K1 cells overexpressing the human adenosine A2a receptor and containing a cAMP inducible beta-galactosidase reporter gene were thawed, centrifuged, DMSO containing media removed, and then seeded with fresh culture media into clear 384-well tissue culture treated plates (BD #353961) at a concentration of 10K cells/well. Prior to assay, these plates were cultured for two days at 37° C., 5% CO₂, 90% Rh. On the day of the functional assay, culture media was removed and replaced with 45 uL assay medium (Hams/F-12 Modified (Mediatech #10-080CV) supplemented w/0.1% BSA). Test compounds were diluted and 11 point curves created at a 1000× concentration in 100% DMSO. Immediately after addition of assay media to the cell plates, 50 nL of the appropriate test compound antagonist or agonist control curves were added to cell plates using a Cartesian Hummingbird. Compound curves were allowed to incubate at room temperature on cell plates for approximately 15 minutes before addition of a 15 nM NECA (Sigma E2387) agonist challenge (5 uL volume). A control curve of NECA, a DMSO/Media control, and a single dose of Forskolin (Sigma F3917) were also included on each plate. After additions, cell plates were allowed to incubate at 37° C., 5% CO₂, 90% Rh for 5.5-6 hours. After incubation, media was removed, and cell plates were washed 1× 50 uL with DPBS w/o Ca & Mg (Mediatech 21-031-CV). Into dry wells, 20 uL of 1× Reporter Lysis Buffer (Promega E3971 (diluted in dH₂O from 5× stock)) was added to each well and plates frozen at −20° C. overnight. For β-galactosidase enzyme calorimetric assay, plates were thawed out at room temperature and 20 μL 2× assay buffer (Promega) was added to each well. Color was allowed to develop at 37° C., 5% CO₂, 90% Rh for 1-1.5 h or until reasonable signal appeared. The calorimetric reaction was stopped with the addition of 60 μL/well 1M sodium carbonate. Plates were counted at 405 nm on a SpectraMax Microplate Reader (Molecular Devices). Data was analyzed in Microsoft Excel and IC/EC50 curves were fit using a standardized macro.

Adenosine A1 Receptor Functional Assay (A1GAL2)

To initiate the functional assay, cryopreserved CHO-K1 cells overexpressing the human adenosine A1 receptor and containing a cAMP inducible beta-galactosidase reporter gene were thawed, centrifuged, DMSO containing media removed, and then seeded with fresh culture media into clear 384-well tissue culture treated plates (BD #353961) at a concentration of 10K cells/well. Prior to assay, these plates were cultured for two days at 37+ C., 5% CO₂, 90% Rh. On the day of the functional assay, culture media was removed and replaced with 45 uL assay medium (Hams/F-12 Modified (Mediatech #10-080CV) supplemented w/0.1% BSA). Test compounds were diluted and 11 point curves created at a 1000× concentration in 100% DMSO. Immediately after addition of assay media to the cell plates, 50 nL of the appropriate test compound antagonist or agonist control curves were added to cell plates using a Cartesian Hummingbird. Compound curves were allowed to incubate at room temperature on cell plates for approximately 15 minutes before addition of a 4 nM r-PIA (Sigma P4532)/1 uM Forskolin (Sigma F3917) agonist challenge (5 uL volume). A control curve of r-PIA in 1 uM Forskolin, a DMSO/Media control, and a single dose of Forskolin were also included on each plate. After additions, cell plates were allowed to incubate at 37° C., 5% CO₂, 90% Rh for 5.5-6 hours. After incubation, media was removed, and cell plates were washed 1× 50 uL with DPBS w/o Ca & Mg (Mediatech 21-031-CV). Into dry wells, 20 uL of 1× Reporter Lysis Buffer (Promega E3971 (diluted in dH₂O from 5× stock)) was added to each well and plates frozen at −20° C. overnight. For β-galactosidase enzyme calorimetric assay, plates were thawed out at room temperature and 20 μL 2× assay buffer (Promega) was added to each well. Color was allowed to develop at 37° C., 5% CO₂, 90% Rh for 1-1.5 h or until reasonable signal appeared. The calorimetric reaction was stopped with the addition of 60 μL/well 1M sodium carbonate. Plates were counted at 405 nm on a SpectraMax Microplate Reader (Molecular Devices). Data was analyzed in Microsoft Excel and IC/EC50 curves were fit using a standardized macro.

A2a ASSAY DATA

| Example | A2AGAL2 Ki μM | A2A-B Ki μM | A1GAL2 Ki μM |
|---|---|---|---|
| 1 | ND | ND | ND |
| 2 | 0.0791955 | ND | 0.467843 |
| 3 | 0.0472389 | ND | 0.437825 |
| 4 | ND | ND | ND |
| 5 | ND | ND | ND |
| 6 | ND | ND | ND |
| 7 | ND | ND | ND |
| 8 | ND | ND | ND |
| 9 | 0.00925337 | 0.0120893 | 0.168345 |
| 10 | 0.0144145 | 0.0341979 | 0.101368 |
| 11 | 0.0293157 | ND | 0.153003 |
| 12 | 0.200909 | ND | 1.28086 |
| 13 | 0.171435 | ND | 0.444939 |
| 14 | 1.03657 | ND | 1.29181 |
| 15 | 0.00503849 | 0.0342768 | 0.15153 |
| 16 | 0.00571479 | ND | 0.0486855 |
| 17 | 0.031989 | ND | 0.277077 |
| 18 | ND | ND | ND |
| 19 | ND | ND | ND |
| 20 | 0.0453002 | ND | 0.292012 |
| 21 | 0.0123055 | 0.043843 | 0.412477 |
| 22 | 0.0252639 | ND | >0.610098 |
| 23 | ND | ND | ND |
| 24 | ND | ND | ND |
| 25 | ND | ND | ND |
| 26 | ND | ND | ND |
| 27 | 0.0397832 | ND | 0.592243 |
| 28 | 0.0874984 | ND | 0.786864 |
| 29 | 0.0050851 | ND | 0.14471 |
| 30 | 0.0162855 | 0.0619156 | 0.347296 |
| 31 | 0.0159845 | ND | 0.285299 |
| 32 | 0.0175752 | ND | 0.408319 |
| 33 | 0.00444325 | 0.00666346 | 0.0842364 |
| 34 | 0.00124222 | 0.0201697 | 0.0260795 |
| 35 | 0.0113763 | 0.0283596 | 0.713181 |
| 36 | 0.155955 | ND | 0.725939 |
| 37 | 0.0643428 | ND | 0.506524 |
| 38 | 0.00141286 | 0.057783 | 0.0300469 |
| 39 | 0.0529907 | ND | 0.289001 |
| 40 | 0.442181 | ND | 0.4432 |
| 41 | ND | ND | ND |
| 42 | ND | ND | ND |
| 43 | ND | ND | ND |
| 44 | ND | ND | ND |

ND indicates no data was available.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

All publications disclosed in the above specification are hereby incorporated by reference in full.

1. A compound of Formula Z 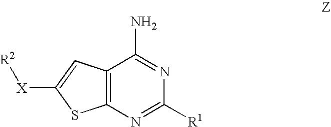
wherein:
X is selected from the group consisting of: 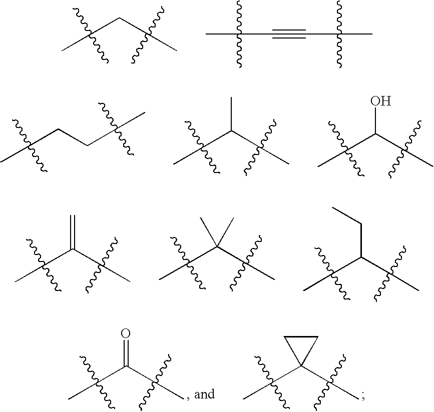
R¹ is phenyl wherein said phenyl is optionally substituted with up to three substituents independently selected from the group consisting of F, Cl, Br, and OCH₃, or a single substituent selected from the group consisting of: OH, OCH₂CF₃, OC₍₁₋₄₎alkyl, C₍₁₋₄₎alkyl, CHF₂, OCF₃, CF₃, and CN;
R² is heteroaryl wherein said heteroaryl is optionally substituted with Cl, F, Br, OC₍₁₋₄₎alkyl, OCF₃, OH, C₍₁₋₄₎alkyl, CHF₂, CF₃, OCH₂CF₃, or a ring selected from the group consisting of: 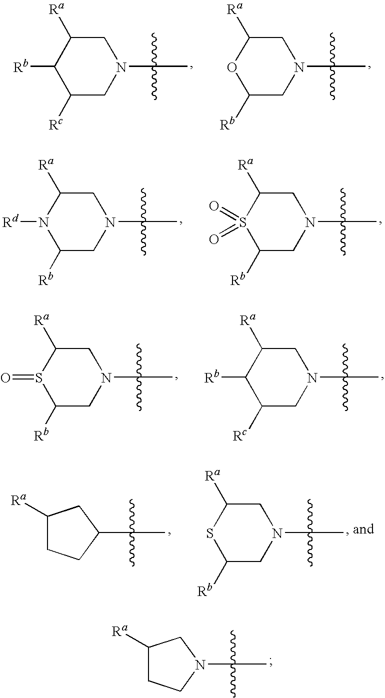
wherein R^{a}, R^{b}, and R^{c} are independently H or C₍₁₋₄₎alkyl;
R^{d} is H, —C₍₁₋₄₎alkyl, —CH₂CH₂OCH₂CH₂OCH₃, —CH₂CO₂H, —C(O)C₍₁₋₄₎alkyl, or —CH₂C(O)C₍₁₋₄₎alkyl;
and solvates, hydrates, tautomers and pharmaceutically acceptable salts thereof.

2. A compound of claim 1, wherein:
R¹ is phenyl, optionally substituted with CN, CF₃, OC₍₁₋₄₎alkyl, OCF₃, C₍₁₋₄₎alkyl, OCH₂CF₃, or up to 3 halogens, selected from the group consisting of Cl, and F;
R² is selected from the group consisting of furyl, imidazolyl, pyrrolyl, oxazolyl, isoxazolyl, pyridinyl, pyrimidinyl, and pyridazinyl, wherein said pyridinyl, pyrimidinyl, and pyridazinyl are optionally substituted with Cl, F, Br, OC₍₁₋₄₎alkyl, OCF₃, piperidinyl, 6-methylpiperidinyl, 3-methylpyrrolidinyl, pyrrolidinyl, morpholinyl, or OCH₂CF₃:
and solvates, hydrates, tautomers and pharmaceutically acceptable salts thereof.

3. A compound of claim 2, wherein
R¹ is phenyl, optionally substituted with CN, CF₃, OC₍₁₋₄₎alkyl, OCF₃, C₍₁₋₄₎alkyl, or up to 3 halogens, selected from the group consisting of Cl, and F;
R² is selected from the group consisting of pyridinyl, pyrimidinyl, and pyridazinyl, wherein said pyridinyl, pyrimidinyl, and pyridazinyl are optionally substituted with Cl, F, Br, OC₍₁₋₄₎alkyl, piperidinyl, pyrrolidinyl, morpholinyl, or OCH₂CF₃:
and solvates, hydrates, tautomers and pharmaceutically acceptable salts thereof.

4. A compound of claim 3, wherein:
R¹ is phenyl, optionally substituted with CN, CF₃, or up to 3 halogens, selected from the group consisting of Cl, and F;
R² is selected from the group consisting of pyridinyl, and pyridazinyl, wherein said pyridinyl is optionally substituted with Cl, F, Br, OC₍₁₋₄₎alkyl, piperidinyl, pyrrolidinyl, morpholinyl, or OCH₂CF₃:
and solvates, hydrates, tautomers and pharmaceutically acceptable salts thereof.

5. A compound of claim 4, wherein:
X is selected from the group consisting of: 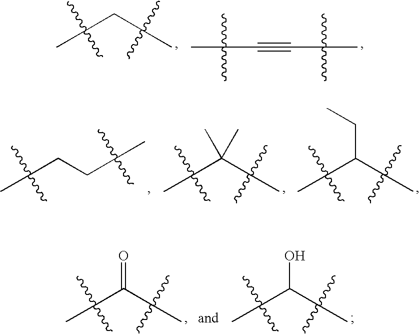
R¹ is phenyl, optionally substituted with CN, or F;
R² is selected from the group consisting of: 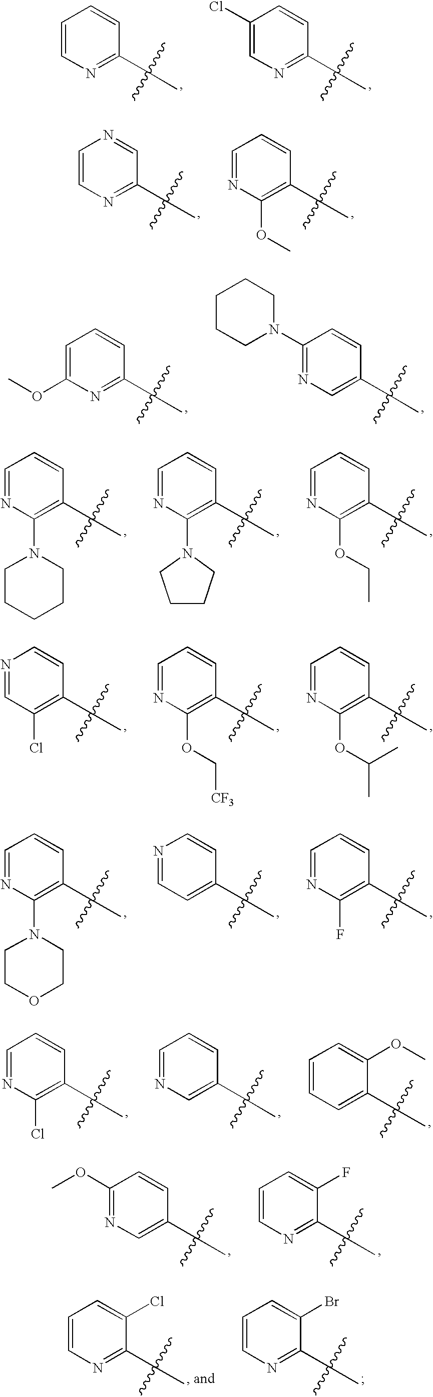
and solvates, hydrates, tautomers and pharmaceutically acceptable salts thereof.

6. A compound selected from the group consisting of: 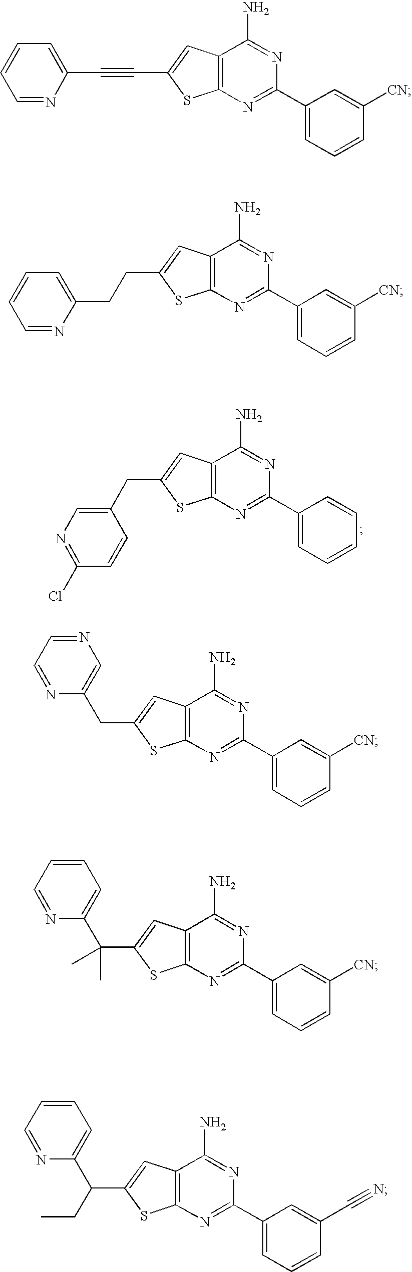 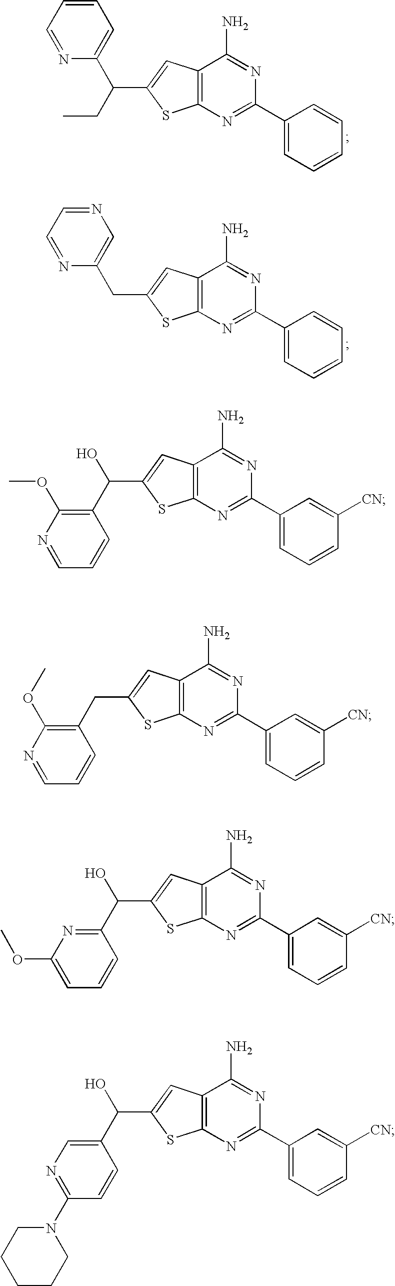 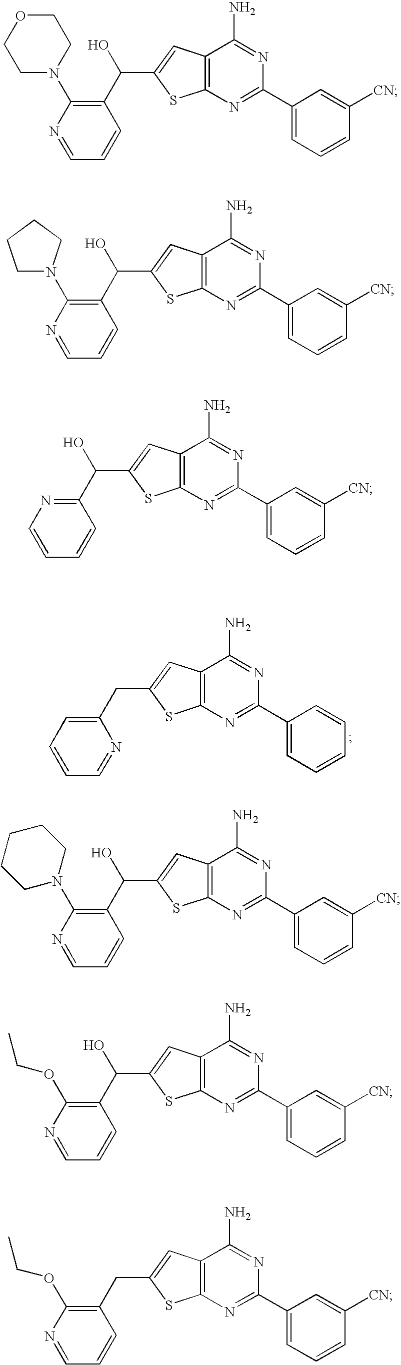 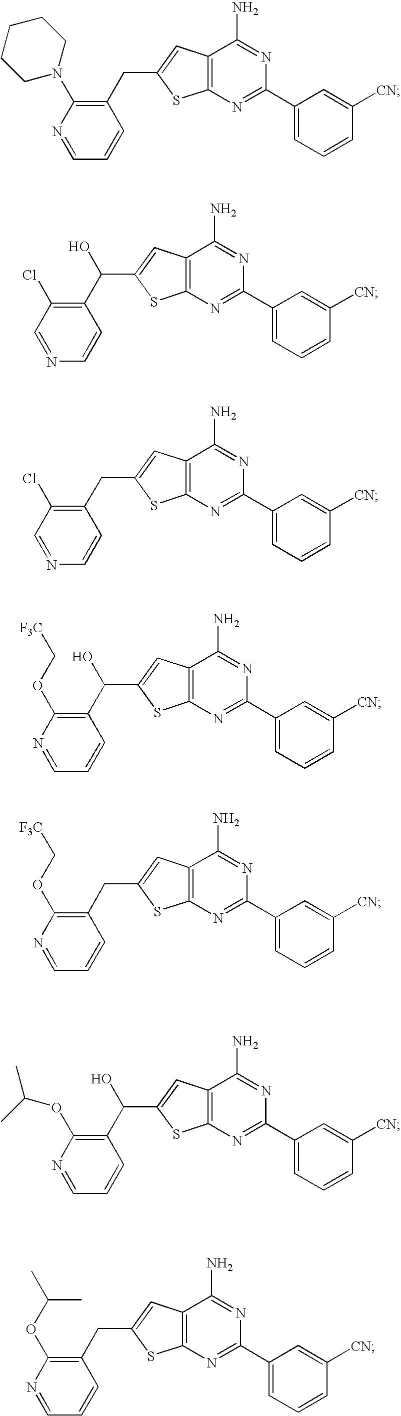 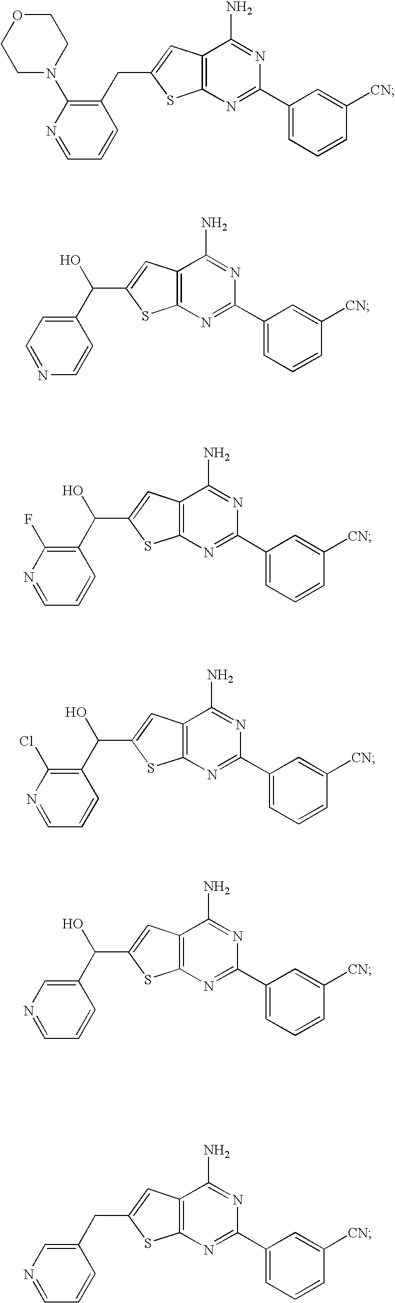 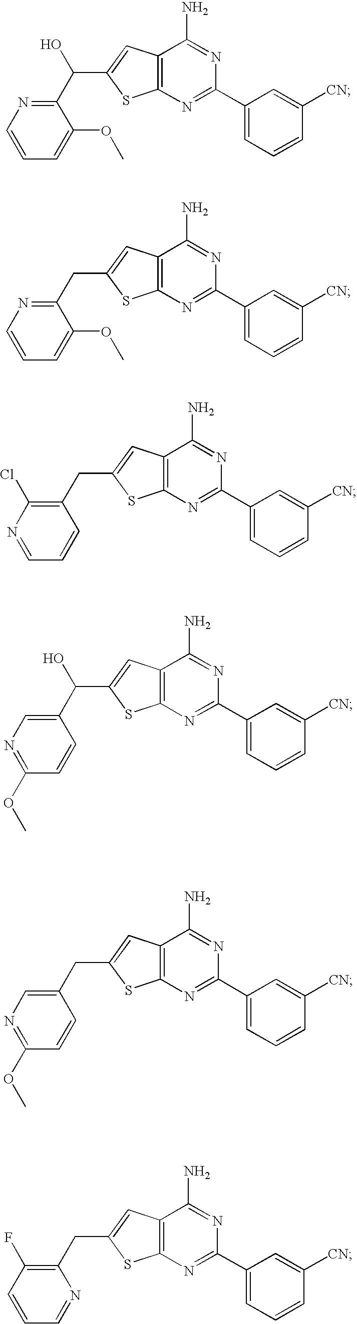 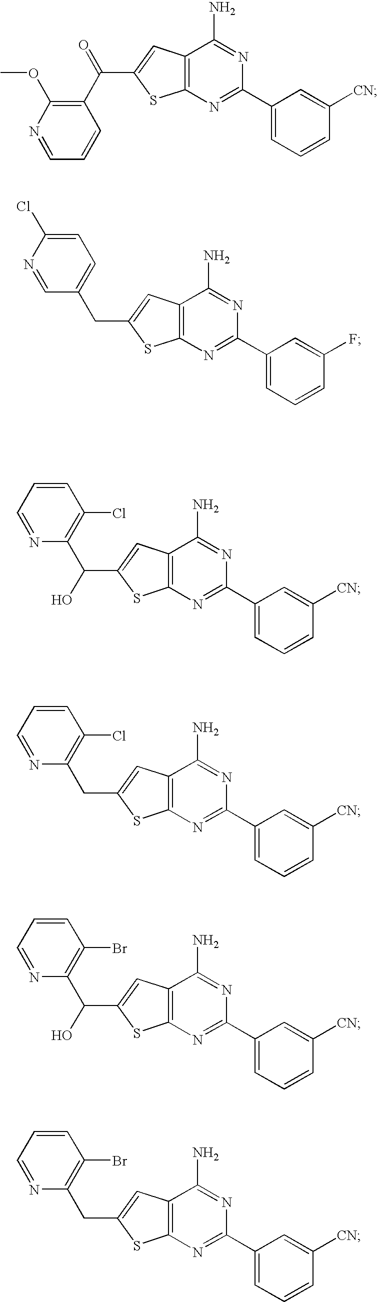
and solvates, hydrates, tautomers and pharmaceutically acceptable salts thereof.

7. A pharmaceutical composition comprising the compound of claim 1; and a pharmaceutically acceptable carrier.

8. A method of treating a subject having a disorder ameliorated by antagonizing Adenosine A2a receptors in appropriate cells in the subject, which comprises administering to the subject a therapeutically effective dose of the compound of claim 1.

9. A method of preventing a disorder ameliorated by antagonizing Adenosine A2a receptors in appropriate cells in the subject, comprising administering to the subject a prophylactically effective dose of the compound of claim 1 either preceding or subsequent to an event anticipated to cause a disorder ameliorated by antagonizing Adenosine A2a receptors in appropriate cells in the subject.

10. The method of treating a subject having a disorder ameliorated by antagonizing Adenosine A2a receptors in appropriate cells in the subject, comprising administering to the subject a therapeutically or prophylactically effective dose of the pharmaceutical composition of claim 7.

11. The method of preventing a disorder ameliorated by antagonizing Adenosine A2a receptors in appropriate cells in the subject, comprising administering to the subject a therapeutically or prophylactically effective dose of the pharmaceutical composition of claim 7.

12. The method of claim 8, wherein the disorder is a neurodegenerative disorder or a movement disorder.

13. The method of claim 8, wherein the disorder is selected from the group consisting of Parkinson's Disease, Huntington's Disease, Multiple System Atrophy, Corticobasal Degeneration, Alzheimer's Disease, and Senile Dementia.

14. The method of claim 9, wherein the disorder is a neurodegenerative disorder or a movement disorder.

15. The method of claim 9, wherein the disorder is selected from the group consisting of Parkinson's Disease, Huntington's Disease, Multiple System Atrophy, Corticobasal Degeneration, Alzheimer's Disease, and Senile Dementia.

16. The method of claim 8, wherein the disorder is Parkinson's Disease.

17. The method of claim 8, wherein the disorder is addiction.

18. The method of claim 8, wherein the disorder is Attention Deficit Hyperactivity Disorder (ADHD).

19. The method of claim 8, wherein the disorder is depression.

20. The method of claim 8, wherein the disorder is anxiety.
